(19) United States
(12) Patent Application Publication
(10) Pub. No.: US 2008/0044353 A1
(43) Pub. Date: Feb. 21, 2008

(54) Combination Therapy for Weight Management

(76) Inventors: James E. Krause, Madision, CT (US); Dario Doller, Sparta, NJ (US); Elena Beretta, Groton, CT (US); Lavanya Rajachandran, Cheshire, CT (US); Robbin Brodbeck, Madison, CT (US)

Correspondence Address: CANTOR COLBURN, LLP, 55 GRIFFIN ROAD SOUTH, BLOOMFIELD, CT, 06002, US

(21) Appl. No.: 11/547,292

(22) PCT Filed: Mar. 28, 2005
(86) PCT No.: PCT/US05/10325

§ 371 Date: Apr. 17, 2007

Related U.S. Application Data
(60) Provisional application No. 60/557,988, filed on Mar. 31, 2004.

(51) Int. Cl. A61K 31/496; A61P 1/00; G01N 33/15

(52) U.S. Cl. 424/9.2; 514/252.12

(57) ABSTRACT

Compositions and methods are provided for weight management. The compositions generally comprise a MCHR antagonist, either in combination with a CB1 antagonist or formulated for coadministration with a CB1 antagonist. Certain methods involve coadministering a MCHR antagonist and a CB1 antagonist to a patient.

### FIELD OF THE INVENTION This invention relates generally to pharmaceutical compositions and therapeutic methods for use in weight management.

### BACKGROUND OF THE INVENTION

Obesity is the most common nutritional problem in developed countries. By some estimates, obesity affects more than half of the population of the United States, where about 300,000 deaths annually are attributable to this condition. Obesity often leads to serious health conditions, such as diabetes, atherosclerosis, pulmonary embolism, coronary artery disease, hypertension, stroke, diabetes, sleep apnea, deep-vein thrombosis, hyperlipidemia and some cancers, and complicates numerous chronic conditions such as respiratory diseases, osteoarthritis, osteoporosis, gall bladder disease and dyslipidemias. Fortunately, many of the conditions caused or exacerbated by obesity can be resolved or dramatically improved by weight loss.

Once considered merely a behavioral problem (i.e., the result of voluntary hyperphagia), obesity is now recognized as a complex multifactorial disease involving defective regulation of food intake, food-induced energy expenditure and the balance between lipid and lean body anabolism. Both environmental and genetic factors play a role in the development of obesity. As a result, treatment programs that focus entirely on behavior modification have limited efficacy and are associated with recidivism rates exceeding 95%. Pharmacotherapy is now seen as a critical component of weight loss and subsequent weight management.

One signaling pathway that contributes to obesity is modulated by melanin concentrating hormone (MCH), a cyclic 19 amino acid hypothalamic peptide. MCH functions as a regulator of food intake and energy balance, serving as a neurotransmitter in the lateral and posterior hypothalamus. MCH activity is mediated by binding to specific receptors, of which MCH type 1 (MCHR1) and type 2 (MCHR2) have been identified. MCHR1 was initially reported by Kolakowski et al. (1996) FEBS Lett. 398:253-58; Lakaye et al. (1998) Biochim. Biophys. Acta 1401:216-220; Chambers et al. (1999) Nature 400:261-65; and Saito et al. (1999) Nature 30 400:265-69. MCHR2 has been described by An et al. (2001) Proc. Natl. Acad. Sci. USA 98:7576-7581; Sailer et al. (2001) Proc. Natl. Acad. Sci. USA 98:7564-7569; Hill et al. (2001) J. Biol. Chem. 276:20125-20129; and Mori et al. (2001) Biochem. Biophys. Res. Commun. 283:1013-1018, and has an overall amino acid identity of more than 30% with MCHR1. Upon binding MCH, MCHR1 and MCHR2 mediate a dose dependent release of intracellular calcium. A variety of MCH receptor antagonists have been reported for use as anti-obesity agents, including those described in PCT International Publication Numbers WO 03/059289, WO 03/060475, WO 02/094799 and WO 02/04433.

Although MCH receptor antagonists show promise in the treatment of obesity, there remains a need in the art for improved pharmaceutical compositions and methods for reducing appetite and for weight management (e.g., treating and preventing obesity and other eating disorders). The present invention fulfills this need, and provides further related advantages.

### SUMMARY OF THE INVENTION

The present invention provides compositions and methods useful for weight management (e.g., the treatment and prevention of obesity), appetite reduction and the treatment of eating disorders.

Within certain aspects, compositions provided herein comprise a first therapeutically effective amount of at least one nontoxic MCHR antagonist and a second therapeutically effective amount of at least one nontoxic CB1 antagonist, together with a physiologically acceptable carrier or excipient.

Packaged pharmaceutical preparations are also provided. Certain such packaged pharmaceutical preparations comprise (i) a container holding a composition comprising at least one nontoxic MCHR antagonist; and (ii) instructions indicating that a first therapeutically effective amount of the MCHR antagonist is to be administered to a patient contemporaneously with a second therapeutically effective amount of a nontoxic CB1 antagonist.

Other packaged pharmaceutical preparations comprise: (i) at least one nontoxic MCHR antagonist; (ii) at least one nontoxic CB1 antagonist and (iii) instructions indicating that a first therapeutically effective amount of the MCHR antagonist(s) and a second therapeutically effective amount of the CB1 antagonist(s) are to be administered to a patient for the treatment of obesity or to reduce food intake and/or appetite.

Within further aspects, methods are provided for reducing food intake and/or appetite in a patient, comprising contemporaneously administering to a patient (i) a first therapeutically effective amount of at least one nontoxic MCHR antagonist and (ii) a second therapeutically effective amount of at least one nontoxic CB1 antagonist.

Within further aspects, methods are provided for treating obesity in a patient, comprising contemporaneously administering to a patient (i) a first therapeutically effective amount of at least one nontoxic MCHR antagonist and (ii) a second therapeutically effective amount of at least one nontoxic CB1 antagonist.

Within certain embodiments of the compositions, packaged pharmaceutical preparations and methods provided herein, the first therapeutically effective amount is less than ½ the maximum recommended for the MCHR antagonist and/or the second therapeutically effective amount is less than ½ the maximum recommended dose for the CB1 antagonist.

Within other embodiments of the compositions, packaged pharmaceutical preparations and methods provided herein, the first therapeutically effective amount is less than the minimum dose of the MCHR antagonist proven effective in a United States clinical trial of the MCHR antagonist without coadministration of a second anti-obesity agent and/or the second therapeutically effective amount is less than the minimum dose of the CB1 antagonist proven effective in a United States clinical trial of the CB1 antagonist without coadministration of a second anti-obesity agent.

Within still further embodiments of the packaged pharmaceutical preparations and methods provided herein, the first therapeutically effective amount is less than the minimum marketed dose of the MCHR antagonist for administration to the patient without coadministration of a second anti-obesity agent and/or the second therapeutically effective amount is less than the minimum marketed dose of the CB1 antagonist for administration to the patient without coadministration of a second anti-obesity agent.

Methods are further provided, within other aspects, for identifying active ingredients for a pharmaceutical composition that comprises a MCHR antagonist and a CB1 antagonist, or for selecting therapeutic agents for coadministration to a patient, such methods comprising selecting a MCHR antagonist and a CB1 antagonist that exhibit at least an additive effect on food intake in a mammal when administered contemporaneously to the mammal in therapeutically effective amounts.

In related aspects, the present invention provides methods for preparing a pharmaceutical composition, comprising the steps of: (i) identifying a MCHR antagonist and a CB1 antagonist that have at least an additive effect on food intake in a mammal when administered contemporaneously to the mammal in therapeutically effective amounts; and (ii) combining a first therapeutically effective amount of the MCHR antagonist and a second therapeutically effective amount of the CB1 antagonist with a physiologically acceptable carrier or excipient.

Also provided herein are methods for preparing a packaged pharmaceutical preparation, comprising the steps of: (i) identifying a MCHR antagonist and a CB1 antagonist that have at least an additive effect on food intake in a mammal when administered contemporaneously to the mammal in therapeutically effective amounts; and (ii) packaging the MCHR antagonist and the CB1 antagonist with instructions indicating that a first therapeutically effective amount of MCHR antagonist and a second therapeutically effective amount of CB1 antagonist are to be administered contemporaneously to a patient for reducing food intake or appetite or for treating obesity.

Methods are further provided for identifying active ingredients for a pharmaceutical composition that comprises a MCHR antagonist and a CB1 antagonist, such methods comprising the steps of: (i) determining an effect of a MCHR antagonist on food intake in a mammal treated with a first therapeutically effective amount of the MCHR antagonist without coadministration of CB1 antagonist; (ii) determining an effect of a CB1 antagonist on food intake in a mammal treated with a second therapeutically effective amount of the CB1 antagonist without coadministration of MCHR antagonist; (iii) determining an effect of the MCHR antagonist and the CB1 antagonist on food intake in a mammal treated contemporaneously with the first therapeutically effective amount of the MCHR antagonist and the second therapeutically effective amount of the CB1 antagonist; (iv) optionally repeating steps (i)-(iii) with a different MCHR antagonist or a different CB1 antagonist; and (v) selecting a MCHR antagonist and a CB1 antagonist for which the effect on food intake determined in step (iii) is equal to or greater than the sum of the effects on food intake determined in steps (i) and (ii);

Methods are further provided, within other aspects, for determining CB1 antagonist activity of a test compound, comprising: (a) contacting a first cell membrane preparation comprising CB1 with: (i) labeled GTP; (ii) a CB1 agonist; and (iii) a test compound; to yield a test membrane preparation; (b) contacting a second cell membrane preparation comprising CB1 with: (i) labeled GTP; and (ii) a CB1 agonist; to yield a control membrane preparation; wherein steps (a) and (b) are performed simultaneously or in either order and under conditions suitable for GTP binding to the CB1; (c) detecting, simultaneously or in either order: (i) a test signal that represents an amount of bound, labeled GTP in the test membrane preparation; and (ii) a control signal that represents an amount of bound, labeled GTP in the control membrane preparation; and (d) comparing the test signal with the control signal. Such methods may be used to identify new CB1 antagonists, as well as to characterize the activity of known CB1 antagonists.

These and other aspects of the present invention will become apparent upon reference to the following detailed description.

### BRIEF DESCRIPTION OF THE DRAWING

FIG. 1 is a graph illustrating the inhibition of excess food consumption by coadministration of a MCHR antagonist and a CB1 antagonist in rats. The results are presented as food consumption in grams during a 12 hour period following administration of vehicle alone, CB1 antagonist alone, MCHR1 antagonist alone, or CB1 antagonist and MCHR1 antagonist in combination, as indicated. “*” indicates P<0.05 and “**” indicates P<0.001.

### DETAILED DESCRIPTION OF THE INVENTION

As noted above, the present invention provides combination therapy for appetite reduction and weight management. In certain aspects, the present invention provides compositions and methods for preventing or treating obesity and/or for reducing appetite or food intake. Such compositions and methods may be used to prevent the development of obesity in a nonobese patient who is at risk for becoming obese (e.g., a previously obese patient), or to facilitate weight reduction in an obese patient. Certain compositions provided herein comprise a nontoxic MCHR antagonist and a nontoxic CB1 antagonist, in combination with a physiologically acceptable carrier or excipient. Methods provided herein generally comprise the coadministration of therapeutically effective amounts of a MCHR antagonist and a CB1 antagonist to a patient.

Terminology

A “patient” is any individual treated, or being considered for treatment, as described herein. Patients include humans, as well as other mammals such as companion animals and livestock, and are generally either obese or are at risk for becoming obese.

As used herein, a patient is considered “obese” if the patient's body mass index is greater than 28. Body mass index (BMI) may be readily calculated using the formula:
BMI=(weight in kg)/(Height in meters)²

A patient is considered to be “at risk for becoming obese” if the patient has one or more risk factors that increase the likelihood of obesity. Such risk factors include, for example, previous obesity, familial obesity and treatment with medication that has the side effect of promoting weight gain.

The term “MCH receptor” or “MCHR” refers to a naturally-occurring mammalian (e.g., human, dog, cat or monkey) MCH type 1 or type 2 receptor, such as MCHR1 and MCHR2, described above. SEQ ID NOs:1 and 2 of PCT International Application Publication No. WO 03/060475 recite the DNA and amino acid sequences, respectively, of a Cynomolgus macaque MCHR1.

A “MCH receptor antagonist” or “MCHR antagonist” is a compound that detectably inhibits MCH receptor-mediated signal transduction. Such inhibition may be determined using the representative functional assay provided in Example 3, or using an assay that detects the ability of the compound to inhibit MCH binding to one or more MCH receptors, such as the assay provided in Example 2. The term “MCHR antagonist” encompasses neutral antagonists and inverse agonists. MCHR antagonists for use as described herein are generally nontoxic. Within certain embodiments, a MCHR antagonist has a relatively low molecular weight (e.g., less than 700 amu) and is multi-aryl (i.e., has a plurality of unfused or fused aryl groups) and amino acid free (e.g., is not a peptide). Preferred MCHR antagonists bind specifically and with high affinity to one or more MCH receptors.

An “inverse agonist” is a compound that reduces the activity of MCH receptor below its basal activity level in the absence of added ligand. Inverse agonists may also inhibit the activity of MCH at MCH receptor, and/or may also inhibit binding of MCH to MCH receptor. The ability of a compound to inhibit the binding of MCH to MCH receptor may be measured by a binding assay, such as the binding assay given in Example 2. The basal activity of MCH receptor, as well as the reduction in MCH receptor activity due to the presence of antagonist, may be determined from a calcium mobilization assay, such as the assay of Example 3.

A “neutral antagonist” of MCH receptor is a compound that inhibits the activity of MCH at MCH receptor, but does not significantly change the basal activity of the receptor (e.g., within an assay as described in Example 3 performed in the absence of ligand, MCH receptor activity is reduced by no more than 10%, more preferably by no more than 5%, and even more preferably by no more than 2%; most preferably, there is no detectable reduction in activity). Neutral antagonists may also inhibit ligand binding of ligand to MCH receptor.

A MCHR antagonist binds “specifically” to MCH receptor if it binds to a MCH receptor (total binding minus nonspecific binding) with a Kᵢ that is 10-fold, preferably I00-fold, and more preferably 1000-fold, less than the Kᵢ measured for binding to other G protein-coupled receptors (e.g., neuropeptide Y, dopamine and beta-adrenergic receptors).

A MCHR antagonist binds with “high affinity” if the Kᵢ at a MCH receptor is less than 1 micromolar, preferably less than 500 nanomolar, 100 nanomolar or 10 nanomolar. MCHR antagonists preferably have minimal agonist activity (i.e., induce an increase in the basal activity of the MCH receptor that is less than 5% of the increase that would be induced by one EC₅₀ of MCH, and more preferably have no detectable agonist activity within the assay described in Example 3). MCHR antagonists may be specific for a particular MCH receptor (e.g., type 1 or type 2), or may function at multiple MCH receptors. In certain embodiments, the MCHR antagonists used within the present invention are MCHR1 antagonists.

A “CB1 antagonist” is a compound that detectably inhibits signal transduction mediated by the cannabinoid receptor CB1. Such inhibition may be determined using the representative agonist-induced GTP binding assay provided in Example 8, or using an assay that detects the ability of the compound to inhibit ligand binding to CBI, such as the assay provided in Example 7. The term “CB1 antagonist” encompasses neutral antagonists, inverse agonists and partial agonists. CB1 antagonists that are neutral antagonists reduce the agonist-stimulated GTP binding activity, but do not result in a binding activity that is below baseline (the level of GTP bound by membranes in this assay in the absence of added agonist). CB1 antagonists that are inverse agonists reduce the GTP binding activity of the receptor-containing membranes below baseline, in the absence of added agonist. A CB1 antagonist that elevates GTP binding activity above baseline in the absence of added CP55,940 is characterized as having partial agonist activity.

Preferred CB1 antagonists bind to CB1 with a Kᵢ of 1 μM or less. The Kᵢ may be determined as described in Example 7, herein, or in an assay performed as described by Felder et al. (1998) J. Pharmacol Exp. Ther. 284:291-297. In certain embodiments, the CB1 antagonist is specific for CB1 (i.e., the K; value at the predominantly peripheral cannabinoid receptor CB2 is greater than 1 μM and/or the Kᵢ ratio (CB2/CB1) is at least 100, preferably at least 1000). IC₅₀ values for CB1 antagonists are preferably 1 μM or less in a functional assay performed as described in Example 8, herein, or in the assay described by Felder et al. CB1 antagonists preferably have minimal agonist activity (i.e., induce an increase in the basal activity of CB1 that is less than 10%, preferably less than 5% and more preferably less than 2% of the increase that would be induced by one EC₅₀ of the agonist CP55,940, and more preferably have no detectable agonist activity within the assay described in Example 8). CB1 antagonists for use as described herein are generally non-toxic.

The term “nontoxic” as used herein shall be understood in a relative sense and is intended to refer to any substance that has been approved by the United States Food and Drug Administration (“FDA”) for administration to mammals (preferably humans) or, in keeping with established criteria, is susceptible to approval by the FDA for administration to mammals (preferably humans). In addition, a highly preferred nontoxic compound generally satisfies one or more of the following criteria: (1) does not substantially inhibit cellular ATP production; (2) does not significantly prolong heart QT intervals; (3) does not cause substantial liver enlargement, or (4) does not cause substantial release of liver enzymes.

As used herein, a compound that does not substantially inhibit cellular ATP production is a compound that satisfies the criteria set forth in Example 9, herein. In other words, cells treated as described in Example 9 with 100 μM of such a compound exhibit ATP levels that are at least 50% of the ATP levels detected in untreated cells. In more preferred embodiments, such cells exhibit ATP levels that are at least 80% of the ATP levels detected in untreated cells.

A compound that does not significantly prolong heart QT intervals is a compound that does not result in a statistically significant prolongation of heart QT intervals (as determined by electrocardiography) in guinea pigs, minipigs or dogs upon administration of twice the minimum dose yielding a therapeutically effective in vivo concentration. In certain preferred embodiments, a dose of 0.01, 0.05, 0.1, 0.5, 1, 5, 10, 40 or 50 mg/kg administered parenterally or orally does not result in a statistically significant prolongation of heart QT intervals. By “statistically significant” is meant results varying from control at the p<0.1 level or more preferably at the p<0.05 level of significance as measured using a standard parametric assay of statistical significance such as a student's T test.

A compound does not cause substantial liver enlargement if daily treatment of laboratory rodents (e.g., mice or rats) for 5-10 days with twice the minimum dose that yields a therapeutically effective iii vivo concentration results in an increase in liver to body weight ratio that is no more than 100% over matched controls. In more highly preferred embodiments, such doses do not cause liver enlargement of more than 75% or 50% over matched controls. If non-rodent mammals (e.g., dogs) are used, such doses should not result in an increase of liver to body weight ratio of more than 50%, preferably not more than 25%, and more preferably not more than 10% over matched untreated controls. Preferred doses within such assays include 0.01, 0.05. 0.1, 0.5, 1, 5, 10, 40 or 50 mg/kg administered parenterally or orally.

Similarly, a compound does not promote substantial release of liver enzymes if administration of twice the minimum dose yielding a therapeutically effective ill vivo concentration does not elevate serum levels of ALT, LDH or AST in laboratory rodents by more than 100% over matched mock-treated controls. In more highly preferred embodiments, such doses do not elevate such serum levels by more than 75% or 50% over matched controls. Alternately, a compound “does not promote substantial release of liver enzymes” if, in an in vitro hepatocyte assay, concentrations (in culture media or other such solutions that are contacted and incubated with hepatocytes iii vitro) equivalent to two-fold the minimum ill vivo therapeutic concentration of the compound do not cause detectable release of any of such liver enzymes into culture medium above baseline levels seen in media from matched mock-treated control cells. In more highly preferred embodiments, there is no detectable release of any of such liver enzymes into culture medium above baseline levels when such compound concentrations are five-fold, and preferably ten-fold the minimum in vivo therapeutic concentration of the compound.

A “prodrug” is a compound that may not be a MCH receptor antagonist or CB1 antagonist, but is modified in vivo, following administration to a patient, to produce such an antagonist. For example, a prodrug may be an acylated derivative of a MCHR antagonist or CB1 antagonist. Prodrugs include compounds wherein hydroxy, amine or sulfhydryl groups are bonded to any group that, when administered to a mammalian subject, cleaves to form a free hydroxyl, amino or sulfhydryl group, respectively. Examples of prodrugs include, but are not limited to, acetate, formate, phosphate and benzoate derivatives of alcohol and amine functional groups within a MCHR antagonist or CB1 antagonist.

MCH Receptor Antagonists

As noted above, the present invention provides compositions and methods useful for weight management. Compositions provided herein generally comprise a nontoxic MCHR antagonist as described above. Preferably, MCHR antagonists for use herein detectably inhibit MCH binding to MCHR1 and/or MCHR2 (as determined using a standard in vitro MCH receptor ligand binding assay and/or calcium mobilization assay) at submicromolar concentrations, preferably at nanomolar concentrations, and more preferably at subnanomolar concentrations. In certain preferred embodiments, MCHR antagonists for use herein detectably inhibit MCH binding to MCHR1. References herein to a “MCH receptor ligand binding assay” refer to the standard in vitro receptor binding assay provided in Example 2. Briefly, a competition assay is performed in which a MCH receptor preparation is incubated with labeled (e.g., ¹²⁵I) MCH and unlabeled test compound. Within the assays provided herein, the MCH receptor used is preferably a mammalian MCHR1 or MCHR2, more preferably a human or monkey MCHR1 or MCHR2 . The MCH receptor preparation may be, for example, a membrane preparation from HEK293 cells that recombinantly express a human MCH receptor (e.g., Genbank- Accession No. Z86090), monkey MCHR1 (such as the MCHR1 sequence provided in SEQ ID NO:1 of WO 03/060475), or human MCHR1/human beta-2-adrenergic chimeric receptor.

Incubation with a MCHR antagonist as described in Example 2 results in a decrease in the amount of label bound to the MCH receptor preparation, relative to the amount of label bound in the absence of the antagonist. Preferably, a MCHR antagonist exhibits a Kᵢ at a MCH receptor of less than 1 micromolar, binding specifically and with high affinity to a MCH receptor. More preferably, such a compound exhibits a Kᵢ at a MCH receptor of less than 500 nM, 100 nM, 20 nM or 10 nM, within a MCH receptor ligand binding assay as described in Example 2.

A representative calcium mobilization assay is provided in Example 3. Preferred MCHR antagonists exhibit IC50 values of 1 micromolar or less, more preferably 100 nanomolar or less, 10 nanomolar or less or 1 nanomolar or less within the standard in? vitro MCH receptor mediated calcium mobilization assay provided in Example 3.

In certain embodiments, MCHR antagonists include substituted 1-benzyl-4-aryl piperazine and piperidine analogues, as described within pending U.S. patent application Ser. No. 10/152,189. The corresponding PCT application published as WO 02/094799 on Nov. 28, 2002, and this disclosure is hereby incorporated herein by reference for its teaching of MCHR antagonists (pages 3-5, 20-25 and 74-107) and the preparation thereof (pages 29-42 and 50-73). Certain MCHR antagonists described therein satisfy the formula: 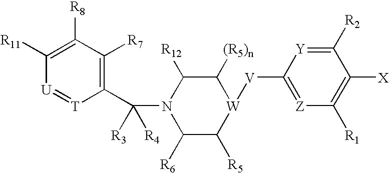
or are a pharmaceutically acceptable salt thereof, wherein:
V is a bond or —C═O)—;
W is nitrogen, CH, COH or CCN;
X is halogen, hydroxy, nitro, cyano, —COOH, oxo and groups of the formula L-M;
Y and Z are each independently: (i) CH, (ii) nitrogen, or (iii) joined with R₅ to form a carbocyclic or heterocyclic ring comprising W and V and having from 5 to 8 ring members, with the proviso that Y and Z are not both nitrogen;
n is 1 or 2;
R₁ and R₂ are each independently selected from: hydrogen, halogen, hydroxy, nitro, cyano, oxo, —COOH and groups of the formula L-M;
R₃ is: (i) selected from hydrogen, (C₁-C₆)alkyl, (C₂-C₆)alkenyl and halo(C₁-C₆)alkyl; or (ii) joined with one or both of R₆ and R₁₀ to form a carbocyclic or heterocyclic group having one ring or two fused rings, wherein each ring contains from 5 to 8 ring members and 0, 1 or 2 heteroatoms independently chosen from oxygen, nitrogen and sulfur;
R₄ is hydrogen, (C₁-C₆)alkyl or halo(C₁-C₆)alkyl;
R₅ is (i) independently selected at each occurrence from hydrogen, halogen, hydroxy, nitro, cyano, amino, oxo, (C₁-C₆)alkyl, (C₂-C₆)alkenyl, (C₂-C₆)alkynyl, (C₁-C₆)alkoxy, halo(C₁-C₆)alkyl, halo(C₁-C₆)alkoxy, mono- and di(C₁-C₆)alkylamino, and amino(C₁-C₆)alkyl; or (ii) joined with R₆, Y or Z to form a carbocyclic or heterocyclic ring having from 5 to 8 ring members;
R₆ is: (i) selected from hydrogen, halogen, hydroxy, nitro, cyano, amino, oxo, (C₁-C₆)alkyl, (C₂-C₆)alkenyl, (C₂-C₆)alkynyl, (C₁-C₆)alkoxy, halo(C₁-C₆)alkyl, halo(C₁-C₆)alkoxy, mono- and di(C₁-C₆)alkylamino, and amino(C₁-C₆)alkyl); or (ii) joined with R₃ or R₅ to form a carbocyclic or heterocyclic group as described above;
R₇ is: (i) selected from hydrogen, halogen, hydroxy, nitro, cyano, —COOH, oxo and groups of the formula L-M; or (ii) joined with R₈ or R₂ to form a fused 5- or 6-membered carbocyclic or heterocyclic group;
R₈ is: (i) selected from hydrogen, halogen, hydroxy, nitro, cyano, —COOH, oxo and groups of the formula L-M; or (ii) joined with R₇ or R₁ to form a fused 5- to 10-member carbocyclic or heterocyclic group;
U is N, O or CR₉;
T is N, O or CR₁₀;
R₉ is: (i) selected from hydrogen, halogen, hydroxy, nitro, cyano, —COOH, oxo and groups of the formula L-M; or (ii) joined with R₁₀ or R₁₁ to form a fused 5- to 10-member carbocyclic or heterocyclic group;
R₁₀ is: (i) selected from hydrogen, halogen, hydroxy, nitro, cyano, —COOH, oxo and groups of the formula L-M; or (ii) joined with R₃, R₈ or R₉ to form a carbocyclic or heterocyclic group;
R₁₁ is: (i) selected from hydrogen, halogen, hydroxy, nitro, cyano, —COOH, oxo and groups of the formula L-M; or (ii) joined with one or both of R₈ and R₉ to form a fused 5- to 10-member carbocyclic or heterocyclic group;
R₁₂ is: (i) independently selected at each occurrence from hydrogen, halogen, hydroxy, nitro, cyano, amino, oxo, (C₁-C₆)alkyl, (C₂-C₆)alkenyl, (C₂-C6)alkynyl, (C₁-C₆)alkoxy, halo(C₁-C₆)alkyl, halo(C₁-C₆)alkoxy, mono- and di(C₁-C₆)alkylamino, and amino(C₁-C₆)alkyl; or (ii) joined with R₇ to form a fused carbocyclic or heterocyclic ring;
L is independently selected at each occurrence from a bond, NR₁₃, O, SO₂, SO₂NH, C(═O)NR₁₃ and NR₁₃C(═O), wherein R₁₃ is independently selected at each occurrence from hydrogen, (C₁-C₆)alkyl, (C₂-C₆)alkenyl, (C₂-C₆)alkynyl, and halo(C₁-C₆)alkyl; and
M is independently selected at each occurrence from hydrogen, (C₁-C₆)alkyl, (C₂-C₆)alkenyl, (C₂-C₆)alkynyl, halo(C₁-C₆)alkyl, amino(C₁-C₆alkyl) and 5 to 10-membered carbocycles.

Representative MCHR antagonists that satisfy the above formula include, for example: 1-(4-bromo-3-methoxyphenyl)-4-(3,4-dimethoxybenzyl) piperazine; 1-(4-bromo-3-methoxy-phenyl)-4-(4-chlorobenzyl)piperazine; 1-[4-chloro-3-(trifluoromethyl)phenyl]-4-(3,4-dimethoxybenzyl) piperazine; 1-(3,4-dichlorophenyl)-4-(3,4-dimethoxybenzyl)piperazine; 1-( 4-bromo-3-methoxyphenyl)-4-(4-methoxy-2,5-dimethylbenzyl)piperazine; 1-(4-bromo-3 -methoxyphenyl)-4-(4-methoxy-2,3-dimethylbenzyl)piperazine; 1-(3-bromo-4-methoxy-benzyl)-4 -(4-bromo-3-methoxyphenyl)piperazine; 4-{[4-(4-bromo-3-methoxy-phenyl)-piperazin-1-yl ]methyl}-2-methoxyphenol; 4-({4-[4-chloro-3-(trifluoromethyl) phenyl]-piperazin-1-yl}methyl)-2-methoxyphenol; 1-[4-chloro-3-(trifluoromethyl) phenyl]-4-(4-methoxy-2,3-dimethylbenzyl)piperazine; 1-(3-bromo-4-methoxybenzyl)-4-[4-chloro-3-(trifluoromethyl) phenyl]piperazine; 1-(4-bromo-3-methoxyphenyl)-4-(4-methoxy-3-methylbenzyl)piperazine; 1-[4 -chloro-3-(trifluoromethyl)phenyl]-4-(4-methoxy-3-methyl-benzyl)piperazine; 1-(4-chloro-3-trifluoromethoxyphenyl)-4-[1-(3,4-dimethoxy-benzyl) ]-2-methyl-piperazine; 4-(4-chloro-3-trifluoromethyl-phenyl)-1-(3,4 -dimethoxy-benzyl)-2-methyl-piperazine; 4-(4-chloro-3-methoxy-phenyl)- -(3,4-dimethoxy-benzyl)-2 -methyl-piperazine; 4-(4-chloro-3-methoxy-phenyl)-[1-(3,4-dimethoxy-benzyl)-ethyl]-2 -methyl-piperazine; 3-{[4-(4-bromo-3-methoxyphenyl)piperazin-1-yl ]methyl}-9-ethyl-9H-carbazole; 1-(5-bromo-6-methoxypyridin-2-yl)-4-(3,4 -dimethoxybenzyl)piperazine; 1-(5-bromo-6-methoxypyridin-2-yl)-4-(4-chlorobenzyl)piperazine; 1 -(5-bromo-6-methoxypyridin-2-yl)-4-(4-methoxy-2,5-dimethylbenzyl) piperazine; 1-(5-bromo-6-methoxypyridin-2-yl)-4-(4-methoxy-2,3 -dimethylbenzyl)piperazine; 1-(3-bromo-4-methoxybenzyl)-4-(5-bromo-6 -methoxypyridin-2-yl)piperazine; 4-(5-bromo-6-methoxypyridin-2-yl)-1-(3,4-dimethoxy-benzyl)-2-methylpiperazine; 4-(5-bromo-6-methoxypyridin-2-yl)-1-(3,4-dimethoxy-benzyl)-2 -methylpiperazine; 1-(5-bromo-6-methoxypyridin-2-yl)-4-(4-methoxy-3-methylbenzyl)piperazine; 3-{[4-(5-bromo-6-methoxypyridin-2-yl)piperazin-1-yl]methyl}-9-ethyl-9H-carbazole; 4-{[4-(5-bromo-6-methoxypyridin-2-yl)piperazin-1-yl]methyl}-2-methoxyphenol; 1-(4-bromo-3-methoxyphenyl)-4-(3,4-dimethoxybenzyl)- 1,4-diazepane;, 1-(4-bromo-3 -methoxyphenyl)-4-[1-(3,4-dimethoxyphenyl)-ethyl]piperazine; 1-[4-chloro-3 -(trifluoromethyl)phenyl]-4-[1-(3,4-dimethoxyphenyl)ethyl]-piperazine; 1-(4-bromo-3 -methoxyphenyl)-4-[1-(4-methoxyphenyl)ethyl]piperazine; 1-(4-chloro-3-methoxyphenyl)-4 -[1-(3,4-dimethoxyphenyl)ethyl]piperazine; 1-(4-bromo-3-methoxyphenyl)-4-[1-(3,4-difluorophenyl)ethyl]piperazine; 4-{1-[4-(4-bromo-3-methoxyphenyl)piperazin-1-yl]ethyl}-2-methylphenol; 1-(4-bromo-3-methoxyphenyl)-4-[1-(4-fluoro-3-methoxyphenyl)-ethyl ]piperazine; 1-(4-chloro-3-methoxyphenyl)-4-[1-(3,4-dimethoxyphenyl)ethyl ]piperazine; 1-(4-chloro-3-methoxyphenyl)4-[1-(3,4-dimethoxyphenyl)ethyl ]piperazine; 1-(4-bromo-3-trifluoromethoxyphenyl)-4-[1-(3-fluoro-4 -methoxyphenyl)ethyl]piperazine; 1-(4-bromo-3-trifluoromethoxyphenyl)-4-[1-(3-fluoro-4-methoxyphenyl)ethyl]piperazine; 1-(4-bromo-3-trifluoromethylphenyl)-4-[1-(3-fluoro-4-methoxyphenyl)ethyl]piperazine; 1-(4-methoxy-phenyl)-4-[1-(3,4-dimethoxyphenyl)ethyl]piperazine; 1-(4 -methoxylphenyl)-4-[1-(3,4-dimethoxyphenyl)ethyl]piperazine; 1-(4 -bromo-3-methoxy-phenyl)-4-[1-(4-chloro-phenyl)-ethyl]-piperazine; I-(4-chloro-3-methoxy-phenyl)-4-[1-(4-methoxy-2,3-dimethyl-phenyl)-ethyl]-piperazine; 1-(4-chloro-3 -methoxy-phenyl)-4-[1-(4-chloro-3-methoxy-phenyl)-ethyl]-piperazine; 1-(4-bromo-3-methoxy-phenyl)-4-[1-(4-methoxy-2,5-dimethyl-phenyl)-ethyl]-piperazine; 1-(4-fluoro-3-trifluoromethyl-phenyl)-4-[1-(3,4-dimethoxy-phenyl)-ethyl]-piperazine; 1-(4-bromo-3-methoxy-phenyl)-4-[1-(3,4-dimethoxy-phenyl)-ethyl]-piperazine; 1-(4-chloro-3-trifluoromethyl-phenyl)-4-[1-(3,4-dimethoxy-phenyl)-ethyl ]-piperazine; 1-(4-bromo-3-methoxy-phenyl)-4-[ 1-(4-ethoxy-3 -methoxy-phenyl)-ethyl]-piperazine; 1-(4-chloro-3-methoxy-phenyl)-4-[1 -(4-fluoro-3-methoxy-phenyl)-ethyl]-piperazine; 1-(4-bromo-3-methoxy-phenyl)-4-[1-(2,4,5-trimethyl-phenyl)-ethyl]-piperazine; 1-(4-bromo-3-methoxy-phenyl)-4 -[1-(3-fluoro-4-methoxy-phenyl)-ethyl]-piperazine; 1-(4-chloro-3 -trifluoromethyl-phenyl)-4-[1-(4-methoxy-2,3-dimethyl-phenyl)-ethyl]-piperazine; 1-(4-fluoro-3-methoxy-phenyl)-4-[1-(3,4-dimethoxy-phenyl)-ethyl]-piperazine; 1-(4 -chloro-3-trifluoromethyl-phenyl)-4-[1-(4-chloro-3-methoxy-phenyl)-ethyl ]-piperazine; 1-(4-chloro-3-trifluoromethyl-phenyl)-4-[1-(4-methoxy-3 -methyl-phenyl)-ethyl]-piperazine; 1-(4-bromo-3-methoxy-phenyl)-4-[1-(3-methoxy-phenyl)-ethyl]-piperazine; 1-(4-bromo-3-methoxy-phenyl)-4-[1-(4-trifluoromethyl-phenyl)-ethyl]-piperazine; 4-(4-fluoro-3-trifluoromethyl-phenyl)-1-[1-(3,4-dimethoxy-phenyl)-ethyl ]-piperazine; 1-(4-chloro-3-methoxy-phenyl)-4-[1-(3-fluoro-4-methoxy-phenyl)-ethyl]-piperazine; 1-(4-bromo-3-methoxy-phenyl)-4-[1-(3-ethoxy-phenyl)-ethyl]-piperazine; 1 -(5-{1-[4-(4-bromo-3-methoxy-phenyl)-piperazin 1-yl]-ethyl}-2 -fluoro-phenyl)-ethanone; 1-(4-chloro-3-methyl-phenyl)-4-[1-(4-methoxy-3 -methyl-phenyl)-ethyl]-piperazine; 1-(4-bromo-3-methoxy-phenyl)-4-[1-(3,5 -dimethoxy-phenyl)-ethyl]-piperazine; 1-(4-methoxy-3-methyl-phenyl)-4-[1-(4-methoxy-2,3-dimethyl-phenyl)-ethyl)- piperazine; 1-(4-chloro-3-methyl-phenyl)-4-[1-(3,4-dimethoxy-phenyl)-ethyl]-piperazine; 1-(4-bromo-3-methoxy-phenyl)-4-[1-(3,4-diethoxy-phenyl)-ethyl]-piperazine; 1-(4-chloro-3-fluoro-phenyl)-4-[1-(4-methoxy-2,3-dimethyl-phenyl)-ethyl]-piperazine; 1-(4-chloro-3-methyl-phenyl)-4-[1-(4-methoxy-2,3-dimethyl-phenyl)-ethyl]-piperazine; 1-(4-chloro-3-fluoro-phenyl)-4-[1-(3,4-dimethoxy-phenyl)-ethyl)-piperazine; 1-(4-methoxy-3-methyl-phenyl)-4-[1-(3-methyl-4-methoxy-phenyl)-ethyl]-piperazine; 1-(4-methoxy-3-methyl-phenyl)-4-[1-(3,4-dimethoxy-phenyl)-ethyl]-piperazine; 1-(3,4-dimethoxy-phenyl)-4-[1-(4-methoxy-2,3-dimethyl-phenyl)-ethyl]-piperazine; 1-(4-fluoro-3-trifluoromethyl-phenyl)-4-[1-(3,4-dimethoxy-phenyl)-ethyl]-piperazine; 1-(4-bromo-3-methoxy-phenyl)-4-{1-[4-(4-bromo-phenyl)-phenyl]-ethyl}-piperazine; 4-(4-chloro-3-trifluoromethyl-phenyl)-[1-(3,4-dimethoxy-benzyl)-ethyl]-piperazine; 1-(1-benzo[1,3]dioxol-5-yl-ethyl)-4-(4-bromo-3-methoxy-phenyl)-piperazine; 1-(4-bromo-3-methoxy-phenyl)-4-[1-(3,4-dimethoxy-phenyl)-ethyl]-[1,4]diazepane; 1-(4-bromo-3-methoxy-phenyl)-4-[1-(3-fluoro-4-methoxy-phenyl)-ethyl]-[1,4]diazepane; 1-[1-(3,4-dimethoxy-phenyl)-ethyl]-4-(3-methoxy-phenyl)-piperazine-2,5-dione; 1-(4-bromo-3-methoxy-phenyl)-4-[1-(3,4-dimethoxy-phenyl)-propyl]-piperazine; 1-(4-chloro-3-trifluoromethyl-phenyl)-4-[1-(3,4-dimethoxy-phenyl)-propyl]-piperazine; 1-(5-bromo-6-methoxypyridin-2-yl)-4-[1-(3,4-dimnethoxyphenyl)ethyl]piperazine; 1-(5-bromo-6-methoxy-pyridin-2-yl)-4-[1-(4-trifluoromethyl-phenyl)-ethyl]-piperazine; 1-(4-bromo-3-methoxy-phenyl)-4-[1-(6-methoxy-naphthalen-2-yl)-ethyl]-piperazine; 1-(4-chloro-3-methoxy-phenyl)-4-[1-(3,4-dimethoxy-phenyl)-ethyl]-piperazine; 1-(4-bromo-3-methoxy-phenyl)-4-[1-(4-fluoro-3-methoxy-phenyl)-ethyl]-piperazine; 1-(4-bromo-3-methoxy-phenyl)-4-[1-(6-methoxy-pyridin-2-yl)-ethyl]-piperazine; 4-(4-chloro-3-trifluoromethyl-phenyl)-1-[1-(3,4-dimethoxy-phenyl)-ethyl]-piperidine; 4-[4-chloro-3-(trifluoromethyl)phenyl]-1-(3,4-dimethoxybenzyl)piperidin-4-ol; 2-(4-chloro-3-methoxy-phenyl)-6-(3,4-dimethoxy-phenyl)-octahydro-pyrido[1 ,2-a]pyrazine; 2-(4-chloro-3-trifluoromethyl-phenyl)-6-(3,4-dimethoxy-phenyl)-octahydro-pyrido[1 ,2-a]pyrazine; 2-(4-chloro-3-methoxy-phenyl)-6-(3-fluoro, 4-methoxy-phenyl)-octahydro-pyrido[1,2-a]pyrazine; 2-(4-fluoro-3-methoxy-phenyl)-6-(3,4-dimethoxy-phenyl)-octahydro-pyrido[1 ,2-a]pyrazine; 2-(4-fluoro-3-trifluoromethyl-phenyl)-6-(3,4-dimethoxy-phenyl)-octahydro-pyrido[1 ,2-a]pyrazine; 2-(4-chloro-3-methoxy-phenyl)-6-methoxy-naphthale12-2-yl)-octahydro-pyrido 1,2-a]pyrazine; 2-(4-chloro-3-methyl-phenyl)-6-(3,4-dimethoxy-phenyl)-octahydro-pyrido[1,2-a]pyrazine; 2-(4-methoxy-3-methyl-phenyl)-6-(3,4-dimethoxy-phenyl)-octahydro-pyrido[1 ,2-a]pyrazine; 2-(4-chloro-3-methoxy-phenyl)-6-(3-methoxy-phenyl)-octahydro-pyrido[1 ,2-a]pyrazine; 2-(2,4-dibromo-5-methoxy-phenyl)-6-(3,4-dimethoxy-phenyl)-octahydro-pyrido[1 ,2-a]pyrazine; 2-(3,4-dimethoxy-phenyl)-6-(3,4-dimethoxy-phenyl)-octahydro-pyrido[1 ,2-a]pyrazine; 2-(4-chloro-3-fluoro-phenyl)-6-(3,4-dimethoxy-phenyl)-octahydro-pyrido[1 ,2-a]pyrazine; 1-(4-fluoro-3-methoxy-phenyl)-6-(3,4-dimethoxy-phenyl)-octahydro-pyrido[1 ,2-a]pyrazine; 2-(4-chloro-3-methoxy-phenyl)-6-(3,4-dimethoxy-phenyl)-octahydro-pyrido[1 ,2-a]pyrazin-8-ol; 2-(4-chloro-3-methoxy-phenyl)-6-(3,4-dimethoxy-phenyl)-octahydro-pyrido[1 ,2-a]pyrazin-S-one; 2-(4-chloro-3-methoxy-phenyl)-6-(3-fluoro-4-methoxy-phenyl)-octahydro-pyrido[1 ,2-a]pyrazin-8-one; 2-(4-chloro-3-methoxy-phenyl)-6-(6-methoxy-naphthalen-2-yl)-octahydro pyrido[1,2-a]pyrazin-8-one; S-(4-chloro-3-methoxy-phenyl)-4-(3,4-dimethoxy-phenyl)-octahydro-pyrazino[2,1-c][1,4]thiazine; 2-(4-chloro-3-methoxy-phenyl)-6-(3,4-dimethoxy-phenyl)-octahydro-pyrrolo[1,2-a]pyrazine; 2-(4-chloro-3-methoxy-phenyl)-6-(3,4-dimethoxy-phenyl)-1,3,4,6,9,9a-hexahydro-2H-pyrido[1 ,2-a]pyrazine; 8-bromo-3-(3,4-dimethoxy-benzyl)-9-methoxy-2,3,4,4a-tetrahydro-1H,6H-pyrazino[1,2-alquinoxalin-5-one; 7-(4-chloro-3-methoxy-phenyl)-4-(3,4-dimethoxy-phenyl)-decahydro-naphthalen-2-ol; 2-(4-chloro-3-methoxy-phenyl)-6-(3,4-dimethoxy-phenyl)-8-fluoro-octahydro-pyrido[1,2-a]pyrazine; 1-(4-bromo-3-methoxyphenyl)-4-(5,6-dimethoxy-2,3-dihydro-1H-indan-1-yl)piperazine; 1-(5-bromo-6-methoxypyridin-2-yl)-4-(5,6-dimethoxy-2,3-dihydro-1H-indan-1-yl)piperazine; 1-(4-chloro-3-trifluoromethyl-phenyl)-4-(4,5-dimethoxy-indan-1-yl)-piperazine; 1-(4-bromo-3-methoxy-phenyl)-4-(4,5-dimethoxy-indan-1-yl)-piperazine; (4-chloro-phenyl)- (4-[1-(2,3-dimethyl-phenyl)-ethyl]-piperazin-1-yl }-methanone; (4-chloro-phenyl)- {4-[1-(4-methoxy-2,3-dimethyl-phenyl)-ethyl]-piperazin-1-yl}-methanone; (4-trifluoro-methyl-phenyl)-(4-[1-(4-methoxy-2,3-dimethyl-phenyl)-ethyl)-piperazin-1-yl }-methanone; (3,4-dichloro-phenyl)-(4-[1-(4-methoxy-2,3-dimethyl-phenyl)-ethyl]-piperazin-1-yl }-methanone; (4-chloro-phenyl)-(4-[1-(4-methyl-naphthalen-1-yl)-ethyl]-piperazin-1-yl}-methanone; (4-trifluoro-methyl-phenyl)-{4-[1-(4-methoxy-2-methyl-phenyl)-ethyl]-piperazin-1-yl}1-methanone; (4-trifluoromethyl-phenyl)- (4-[1-(4-methoxy-2,3-dimethyl-phenyl)-ethyl]-piperazin-1-yl }-methanone; 4-trifluoromethyl-phenyl)- (4-[1-(4-methoxy-3-methyl-phenyl)-ethyl]-piperazin-1-yl}-methanone; (4-chloro-phenyl)- {4-[1-(4-methoxy-naphthalen-1-yl)-ethyl]-piperazin-1-yl}-methanone; 4-trifluoromethyl-phenyl)-{4-[1-(4-methoxy-2,3-dimethyl-phenyl)-propyl]-piperazin-1-yl}-methanone; (4-chloro-phenyl)-(4-[1-(4-methoxy-2,3-dimethyl-phenyl)-allyl]-piperazin-1-yl}-methanone; 4-fluoro-phenyl)-{4-[1-(4-methoxy-2,3-dimethyl-phenyl)-ethyl]-piperazin-1-yl}-methanone; 4-bromno-3-methyl-phenyl)-{4-[1-(4-methoxy-2,3-dimethyl-phenyl)-ethyl]-piperazin-1-yl}-methanone; (3,4-dichloro-phenyl)-{4-[1-(4-methyl-naphthalen-1-yl)-ethyl]-piperazin-1-yl}-methanone; [6-(3,4-dimethoxy-phenyl)-octahydro-pyrido[1 ,2-a]pyrazin-2-yl]-(4-trifluoromethyl-phenyl)-methanone; 4-chloro-phenyl)-{4-[1-(4-methoxy-2,3-dimethyl-phenyl)-propyl]-piperazin-1-yl }-methanone; (4-[1-(4-methoxy-2,3-dimethyl-phenyl)-ethyl]-[1,4]diazepan-1-yl}-(4-trifluoromethyl-phenyl)-methanone; (5-[1-4-methoxy-2,3-dimethyl-phenyl)-ethyl]-2,5-diaza-bicyclo[2,2,1]hept-2-yl}-(4-trifluoromethyl-phenyl)-methanone; 3-(1-[4-(4-bromo-3-methoxy-phenyl)-piperazin-1-yl]-ethyl}-6-methoxy-quinoline; 3-{1-[4-(4-bromo-3-methoxy-phenyl)-piperazin-1-yl]-ethyl}-2-chloro-6-methoxy-quinoline; 3- {1-[4-(4-bromo-3-methoxy-phenyl)-piperazin-1-yl]-ethyl}-quinoline; 1-(4-bromo-3-methoxy-phenyl)-4-[1-(6-methoxy-pyridin-3-yl)-ethyl]-piperazine; 3-{1-[4-(4-bromo-3-methoxy-phenyl)-piperazin-1-yl]-ethyl}-6-fluoro-4-methyl-2H-chromen-2-ol; (3S)-1-(4-chloro-3-trifluoromethoxyphenyl)-4-[1-(3,4-dimethoxy-benzyl))]2-methyl-piperazine; (2S)-4-(5-bromo-6-methoxypyridin-2-yl)-1-(3,4-dimethoxy-benzyl)-2-methylpiperazine; R-1-(4-chloro-3-methoxyphenyl)-4-[1-(3,4-dimethoxyphenyl)ethyl]piperazine; S-1-(4-chloro-3-methoxyphenyl)-4-[1-(3,4-dimethoxyphenyl)ethyl]piperazine; R-1-(4-bromo-3-trifluoromethoxyphenyl)-4-[1-(3-fluoro-4-methoxyphenyl)ethyl]piperazine; S-1-(4-bromo-3-trifluoromethylphenyl)-4-[1-(3-fluoro-4-methoxyphenyl)ethyl]piperazine; S-1-(4-methoxy-phenyl)-4-[1-(3,4-dimethoxyphenyl)ethyl]piperazine; R-1-(4-methoxylphenyl)-4-[1-(3,4-dimethoxyphenyl)ethyl]piperazine; R-1-(4-chloro-3-methoxy-phenyl)-4-[1-(3,4-dimethoxy-phenyl)-ethyl]-piperazine; {5-[1-4-methoxy-2,3-dimethyl-phenyl)-ethyl]-(1S,4S)-2,5-diaza-bicyclo[2,2,1]hept-2-yl}-(4-trifluoromethyl-phenyl)-methanone; (6R, 10S)-2-(4-chloro-3-methoxy-phenyl)-6-(3,4-dimethoxy-phenyl)-octahydro-pyrido[1,2-a]pyrazin-8-one; R-1-(4-bromo-3-methoxyphenyl)-4-[f-(3,4-dimethoxyphenyl)-ethyl]piperazine; (2S)-4-(4-chloro-3-methoxyphenyl)-1-(3,4-dimethoxybenzyl)-2-methyl-piperazine; (2R)-4-(4-chloro-3-methoxyphenyl)-1-(3,4-dimethoxybenzyl)-2-methyl-piperazine; (3R)-1-(4-fluoro-3-trifluoromethyl-phenyl)-4-[1-(3,4-dimethoxyphenyl)-ethyl]-piperazine; (3S)- -(4-fluoro-3-trifluoromethyl-phenyl)-4-[1-(3,4-dimethoxyphenyl)-ethyl]-piperazine; (3R)-1-(4-chloro-3-trifluoromethyl-phenyl)-4-[1-(3,4-dimethoxyphenyl)-ethyl]-piperazine; (3S)-1-(4-chloro-3-trifluoromethyl-phenyl)-4-[1-(3,4-dimethoxyphenyl)-ethyl]-piperazine; (6R, 10S)-2-(4-chloro-3-methoxy-phenyl)-6-(3,4-dimethoxy-phenyl)-octahydro-pyrido[1,2-a]pyrazine; (3R)-1-(4-fluoro-3-methoxy-phenyl)-4-[1-(3,4-dimethoxyphenyl)-ethyl]-piperazine; (2R)-4-(4-chloro-3-trifluoromethylphenyl)-[1-(3,4-dimethoxybenzyl)-ethyl]-piperazine; (6R,9S)-2-(4-chloro-3-methoxy-phenyl)-6-(3,4-dimethoxy-phenyl)-octahydro-pyrrolo[1,2-a]pyrazine; (6R, 10S)-2-(4-chloro-3-methoxy-phenyl)-6-(3,4-dimethoxy-phenyl)-octahydro-pyrido[1,2-a]pyrazin-8-ol; (6R,10S)-[6-(3,4-dimethoxy-phenyl)-octahydro-pyrido[1,2-a]pyrazin-2-yl]-(4-trifluoromethyl-phenyl)-methanone; (6R,10S)-2-(4-chloro-3-methoxy-phenyl)-6-(3-fluoro, 4-methoxy-phenyl)-octahydro-pyrido[1,2-a]pyrazine; and {5-[1-4-methoxy-2,3-dimethyl-phenyl)-ethyl]-(1S,4S)-2,5-diaza-bicyclo[2,2,1]hept-2-yl} -(4-trifluoromethyl-phenyl)-methanone, as well as pharmaceutically acceptable salts of the foregoing.

Within other embodiments, MCHR antagonists for use within the present compositions and methods are substituted benzimidazole analogues as described within pending U.S. patent application Ser. No. 10/399,499, filed Jan. 9, 2003. The corresponding PCT application published as WO 03/060475 on Jul. 24, 2003, and this disclosure is hereby incorporated herein by reference for its teaching of MCHR antagonists (pages 2-5, Table I (pages 14-19) and Table II (pages 38-48) and the preparation thereof (pages 23-24 and 32-38).

Within further embodiments, compounds for use within the present invention are as described within pending U.S. patent application Ser. No. 10/399,111, filed Jan. 9, 2003. The corresponding PCT application published as WO 03/059289 on Jul. 24, 2003, and this disclosure is hereby incorporated herein by reference for its teaching of MCHR antagonists (pages 3-4 and 31-50) and the preparation thereof (pages 19-20 and 28-31). Within further embodiments, MCHR antagonists for use within the present invention are as described within U.S. Pat. No. 6,569,861, which is hereby incorporated by reference for its teaching of phenylcycloallkylmethylamino and phenylalkenylamino MCHR antagonists (columns 3-9 and 18-19) and the preparation thereof (columns 16-18).

Still further MCHR antagonists are described, for example, within the following published PCT applications: WO 03/097047, WO 03/087046, WO 03/087045, WO 03/087044, WO 03/072780, WO 03/070244, WO 03/047568, WO 03/045920, WO 03/045918, WO 03/045313, WO 03/035055, WO 03/033480, WO 03/015769, WO 03/028641, WO 03/013574, WO 03/004027, WO 02/089729, WO 02/083134, WO 02/076947, WO 02/076929, WO 02/057233, WO 02/051809, WO 02/10146, WO 02/06245, WO 02/04433, WO 01/87834,.WO 01/82925, WO 01/57070, WO 01/21577 and WO 01/21169, as well as Japanese Application Publication Number 2001-226269. It will be apparent that the above are illustrative examples of MCHR antagonists, and are not intended to limit the scope of the present invention.

CB1 Antagonists

CB1 is a cannabinoid receptor that is predominantly expressed in the central nervous system and is involved in the regulation of appetite and food consumption. Any CB1 antagonist(s) may be used within the compositions and methods provided herein. In general, a CB1 antagonist for use as described herein should have a high affinity for CB1 (i.e., the KE value at the CB1 receptor is 1 μM or less, preferably 100 nM or less, and more preferably 10 nM or less). In certain embodiments, the CB1 antagonist is specific for CB1 (i.e., the Kᵢ value at CB2 is greater than 1 μM; and/or the Kᵢ ratio (CB2/CB1) is at least 100, preferably at least 1000). IC₅₀ values for CB1 antagonists are preferably 1 μM or less.

Representative CB1 antagonists for use as described herein include, for example, certain pyrimidines (e.g., PCT International Application Publication No. WO 04/029,204), pyrazines (e.g., PCT International Application Publication Nos. WO 01/111,038; WO 04/111,034 and WO 04/111,033), azetidine derivatives (e.g., U.S. Pat. Nos. 6,518,264; 6,479,479 and 6,355,631; and PCT International Application Publication No. WO 03/053431), pyrazole derivatives (e.g., U.S. Pat. Nos. 6,509,367 and 6,476,060; and PCT International Application Publication Nos. WO 03/020217 and WO 01/029007), pyrazolecarboxylic acid and pyrazole carboxamide derivatives (e.g., U.S. Pat. Nos. 6,645,985; 6,432,984; 6,344,474; 6,028,084; 5,925,768; 5,624,941 and 5,462,960; published US applications US 2004/0039024; US 2003/0199536 and US 2003/0003145; and PCT International Application Publication Nos. WO 03/078413; WO 03/027076; WO 03/026648 and WO 03/026647); aroyl substituted benzofurans (e.g., LY-320135, U.S. Pat. No. 5,747,524); substituted imidazoles (e.g., published US application US 2003/0114495 and PCT International Application Publication Nos. WO 03/063781 and WO 03/040107); substituted furo[2,3-b]pyridine derivatives (e.g., PCT International Application Publication No. WO 04/012671); substituted aryl amides (e.g., PCT International Application Publication Nos. WO 03/087037 and WO 03/077847); substituted bicyclic or spirocyclic amides (e.g., PCT International Application Publication Nos. WO 03/086288 and WO 03/082190); and substituted 2,3-diphenyl pyridines (e.g., PCT International Application Publication No. WO 03/082191). Other CB1 antagonists are cannabidiol and its derivatives. Preferred CB 1 antagonists include, for example, aryl substituted pyrazole carboxamides such as SR-141716A (N-piperidin-1-yl)-5-(4-chlorophenyl)-1-(2,4-dichlorophenyl)-4-methyl-1-H-pyrazole-3-carboxamide, also known as RIMONABANT™ or ACOMPLIA™) as well analogues thereof such as AM251. (N-piperidin-1-yl)-5-(4-iodophenyl)-1-(2,4-dichlorophenyl)-4-methyl-1-H-pyrazole-3-carboxamide) and AM281 (N-(morpholin-4-yl)-1-(2,4-dichlorophenyl)-5-(4-iodophenyl)-4-methyl-1-H-pyrazole-3-carboxamide); various azetidine compounds (e.g., U.S. Pat. Nos. 6,518,264; 6,479,479 and 6,355,631) and the imidazoles 1-(4-chlorophenyl)-2-(2-chlorophenyl)-N-[(1S,2S)-2-hydroxycyclohexyl)-1H-imidazole-4-carboxamide and 2-(2-chlorophenyl)-1-(4-chlorophenyl)-N′-[4-(trifluoromethyl)phenyl]-1H-imidazole-4-carbohydrazide.

CB1 antagonists may be identified or characterized using an agonist-induced GTP binding assay, such as the assay described in Example 8, herein. Such assays employ a CB1-containing cell membrane preparation (e.g., a preparation of membranes of insect cells that recombinantly express CB1) to determine the effect of a test compound on CB1 agonist-induced GTP binding to CB1. Briefly, a first cell membrane preparation comprising CB1 is contacted with: (i) labeled GTP; (ii) a CB1 agonist; and (iii) a test compound to yield a test membrane preparation. Simultaneously, or in either order, a second cell membrane preparation comprising CB1 is contacted with: (i) labeled GTP; and (ii) a CB1 agonist to yield a control membrane preparation. The labeled GTP is preferably GTP-gamma³⁵S; a representative CB1 agonist is CP55,940. Such contact is performed under conditions that are suitable for GTP binding to CB1, such as the conditions described in Example 8. The concentrations of labeled GTP and CB1 agonist used are generally concentrations that are sufficient to result in a detectable increase in the amount of labeled GTP bound to the membrane preparation in the presence of CB1 agonist. Such concentrations may be determined by routine experimentation; representative suitable concentrations are provided in Example 8. Generally, a range of test compound concentrations is used (e.g., ranging from 10⁻¹⁰M to 10⁻⁵M).

After sufficient contact (e.g., incubation) to allow GTP binding to the membrane preparations, a signal that corresponds to (represents) the amount of bound, labeled GTP is detected (typically, unbound labeled GTP is first removed via a washing step). In other words, simultaneously or in either order: (i) a test signal that represents an amount of bound, labeled GTP in the test membrane preparation is detected; and (ii) a control signal that represents an amount of bound, labeled GTP in the control membrane preparation is detected. The nature of the signal detected is determined by the type of label used. For example, if the GTP is radioactively labeled, the signal detected is radioactive decay (e.g., via liquid scintillation spectrometry). The CB1 antagonist activity of the test compound is then determined by comparing the test signal with the control signal. A test signal that is lower than the control signal indicates that the test compound is a CB1 antagonist.

Within the compositions and methods described herein, MCHR antagonist(s) and/or CB1 antagonist(s) may be present as a pharmaceutically acceptable salt or a prodrug. As used herein, a “pharmaceutically acceptable salt” is an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recogilize further pharmaceutically acceptable salts, including those listed by Remington's Pharmaceutical Sciences, 17th ed., Mack Publishing Company, Easton, Pa., p. 1418 (1985). Accordingly, the present disclosure should be construed to include all pharmaceutically acceptable salts of MCHR and CB1 antagonists.

A wide variety of synthetic procedures are available for the preparation of pharmaceutically acceptable salts. In general, a pharmaceutically acceptable salt can be synthesized from a parent compound that contains a basic or acidic moiety by any conventional chemical method. Briefly, such salts can be prepared by reacting the free acid or base form of these compounds with a stoichiometric amount of the appropriate base or acid in water or in an organic solvent, or in a mixture of the two; generally, nonaqueous media like ether, ethyl acetate, ethanol, isopropanhol, or acetonitrile are preferred.

Prodrugs of MCHR antagonists and/or CB1 antagonists may be prepared by modifying functional groups present in the compounds in such a way that the modifications are cleaved to the parent compounds. Those of ordinary skill in the art will recognize various synthetic methods that may be employed to prepare prodrugs of a MCHR antagonist or CB1 antagonist, and will appreciate that such prodrugs may be used in place of the active antagonist within the compositions and methods provided herein.

Pharmaceutical Compositions

The practice of the present invention employs pharmaceutical compositions comprising at least one nontoxic MCHR antagonist, together with at least one physiologically acceptable carrier or excipient, as well as pharmaceutical compositions comprising at least one at least one nontoxic CB1 antagonist, together with at least one physiologically acceptable carrier or excipient. In certain aspects, the MCHR antagonist(s) and CB1 antagonist(s) are present within the same pharmaceutical composition; in other aspects, MCHR antagonist(s) are present within one pharmaceutical composition and CB1 antagonist(s) are present within a separate pharmaceutical composition.

Pharmaceutical compositions may comprise, for example, water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. Certain pharmaceutical compositions are formulated for oral delivery to humans or other animals (e.g. companion animals such as dogs).

If desired, other anti-obesity agents may also be included, such as leptin, a leptin receptor agonist, a melanocortin receptor 4 (MC4) agonist, sibutramine, dexfenfluramine, a growth hormone secretagogue, a beta-3 agonist, a 5HT-2 agonist, an orexin antagonist, a neuropeptide Y₁ or Y₅ antagonist, a galanin antagonist, a CCK agonist, a GLP-1 agonist and/or a corticotropin-releasing hormone agonist.

Pharmaceutical compositions may be formulated for any appropriate manner of administration, including, for example, topical, oral, nasal, rectal or parenteral administration. The term parenteral as used herein includes subcutaneous, intradermal, intravascular (e.g., intravenous), intramuscular, spinal, intracranial, intrathecal and intraperitoneal injection, as well as any similar injection or infusion technique. In certain embodiments, compositions in a form suitable for oral use are preferred. Such forms include, for example, tablets, troches, lozenges, aqueous or oily suspensions, dispersible powders or granules, emulsion, hard or soft capsules, or syrups or elixirs. Within yet other embodiments, compositions of the present invention may be formulated as a lyophilizate.

Compositions intended for oral use may further comprise one or more components such as sweetening agents, flavoring agents, coloring agents and preserving agents in order to provide appealing and palatable preparations. Tablets contain the active ingredient in admixture with physiologically acceptable excipients that are suitable for the manufacture of tablets. Such excipients include, for example, inert diluents (e.g., calcium carbonate, sodium carbonate, lactose, calcium phosphate or sodium phosphate), granulating and disintegrating agents (e.g., corn starch or alginic acid), binding agents (e.g., starch, gelatin or acacia) and lubricating agents (e.g., magnesium stearate, stearic acid or talc). The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent (e.g., calcium carbonate, calcium phosphate or kaolin), or as soft gelatin capsules wherein the active ingredient is mixed with water or an oil medium (e.g., peanut oil, liquid paraffin or olive oil).

Aqueous suspensions comprise the active materials in admixture with suitable excipients, such as suspending agents (e.g., sodium carboxymethylcellulose, methylcellulose, hydropropylmethylcellulose, sodium alginate, polyvinylpyrrolidone, gum tragacanth and gum acacia); and dispersing or wetting agents (e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxy ethylene sorbitol monooleate, or condensation products of ethylene oxide with partial esters derived from fatty acids and hexitol anhydrides such as polyethylene sorbitan monooleate). Aqueous suspensions may also contain one or more preservatives, for example ethyl, or n-propyl p-hydroxybenzoate, one or more coloring agents, one or more flavoring agents, and/or one or more sweetening agents such as sucrose or saccharin.

Oily suspensions may be formulated by suspending the active ingredients in a vegetable oil (e.g., arachis oil, olive oil, sesame oil or coconut oil) or in a mineral oil such as liquid paraffin. The oily suspensions may contain a thickening agent such as beeswax, hard paraffin or cetyl alcohol. Sweetening agents such as those set forth above, and/or flavoring agents may be added to provide palatable oral preparations. Such suspension may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavoring and/or coloring agents, may also be present.

Pharmaceutical compositions may also be in the form of oil-in-water emulsions. The oily phase may be a vegetable oil (e.g., olive oil or arachis oil) or a mineral oil (e.g., liquid paraffin) or mixtures thereof. Suitable emulsifying agents may be naturally-occurring gums (e.g., gum acacia or gum tragacanth), naturally-occurring phosphatides (e.g., soy bean, lecithin, and esters or partial esters derived from fatty acids and hexitol), anhydrides (e.g., sorbitan monoleate) and condensation products of partial esters derived from fatty acids and hexitol with ethylene oxide (e.g., polyoxyethylene sorbitan monoleate). The emulsions may also contain sweetening and/or flavoring agents.

Syrups and elixirs may be formulated with sweetening agents, such as glycerol, propylene glycol, sorbitol or sucrose. Such formulations may also comprise one or more demulcents, preservatives, flavoring agents and/or coloring agents.

A pharmaceutical composition may be prepared as a sterile injectible aqueous or oleaginous suspension. The active ingredients, depending on the vehicle and concentration used, can either be suspended or dissolved in the vehicle. Such a composition may be formulated according to the known art using suitable dispersing, wetting agents and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid find use in the preparation of injectible compositions, and adjuvants such as local anesthetics, preservatives and/or buffering agents can be dissolved in the vehicle.

Compositions may also be prepared in the form of suppositories (e.g., for rectal administration). Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

For administration to non-human animals, the composition may also be added to animal feed or drinking water. It may be convenient to formulate animal feed and drinking water compositions so that the animal takes in an appropriate quantity of the composition along with its diet. It may also be convenient to present the composition as a premix for addition to feed or drinking water.

Pharmaceutical compositions may be formulated as sustained release or controlled-release formulations (i.e., a formulation such as a capsule that effects a slow release of MCHR antagonist(s) and/or CB1 antagonist(s) following administration). Such formulations may generally be prepared using well known technology and administered, for example, orally, rectally or by implantation, or by implantation, either subcutaneous or at a desired target site. Carriers for use within such formulations are biocompatible, and may also be biodegradable; preferably the formulation provides a relatively constant level of MCHR antagonist and/or CB1 antagonist release. The amount of antagonist contained within a sustained release formulation depends upon the site of implantation, the rate and expected duration of release and the nature of the condition to be treated or prevented.

For pharmaceutical compositions that are formulated in single-dose units (e.g., tablets or capsules), MCHR antagonist(s) and/or CB1 antagonist(s) are generally present within each unit in a therapeutically effective amount. A therapeutically effective amount (or dose) is an amount that results in a discernible benefit in a patient when the MCHR antagonist(s) and CB1 antagonist(s) are administered contemporaneously and repeatedly at a prescribed frequency (e.g., from 1 to 4 times per day for a period of weeks or months) to a patient. Such benefit(s) include one or more of decreased BMI, decreased appetite or food intake and/or weight loss. A therapeutically effective amount of MCHR antagonist is an amount that results in such a discernible patient benefit when so administered, as compared to the patient benefit observed following administration of CB1 antagonist alone. Similarly, a therapeutically effective amount of CB1 antagonist is an amount that results in such a discernible patient benefit when so administered, as compared to the patient benefit observed following administration of MCHR antagonist alone. “Contemporaneously,” as used herein, refers to a time frame such that the MCHR antagonist is present in a body fluid of a patient (at concentration that is sufficient to alter the binding of MCH to a MCH receptor and/or MCH-mediated signal transduction i n vitro) at the same time as the CB1 antagonist is present in the body fluid (at concentration that is sufficient to detectably alter the binding of CB1 ligand to CB1 and/or CB1-mediated signal transduction in vitro). Contemporaneous administration is also referred to herein as “coadministration.” A preferred concentration of MCHR antagonist in a body fluid of a patient is one sufficient to inhibit the binding of MCH to MCHR1. Compositions providing dosage levels ranging from about 0.1 mg to about 140 mg per kilogram of body weight per day are preferred (about 0.5 mg to about 7 g per human patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. Optimal dosages may be established using routine testing and well known procedures.

In certain embodiments, a therapeutically effective amount of MCHR antagonist is lower than the amount that would need to be administered to effect a comparable patient benefit in the absence of CB1 antagonist. Within certain compositions and methods provided herein, at least an additive effect is observed (i.e., the patient benefit is at least the sum of the benefits that would be achieved by the separate administration of the same amounts of MCHR antagonist and CB 1 antagonist).

In certain embodiments, the therapeutically effective amount of MCHR antagonist is less than ¾, ½, ¼ or 10% of the maximum recommended dose for the MCHR antagonist (i.e., the maximum dose advised by the manufacturer or the U.S. Food and Drug Administration (FDA)). Similarly, in certain embodiments, a therapeutically effective amount of CB1 antagonist is lower than the amount that would need to be administered to effect a comparable patient benefit in the absence of MCHR antagonist. In certain embodiments, the therapeutically effective amount of CB1 antagonist is less than ¾, ½, ¼ or 10% of the maximum recommended dose for the CB1 antagonist (i.e., the maximum dose advised by the manufacturer or the FDA).

In further embodiments, the therapeutically effective amount of MCHR antagonist is less than the minimum dose of the MCHR antagonist proven effective in a United States clinical trial of the MCHR antagonist, wherein the trial is conducted without coadministration of a second anti-obesity agent, such as a CB1 antagonist (e.g., the therapeutically effective amount is less than 95%, less than 90%, less than 75% or less than 50% of the minimum dose proven effective in such a clinical trial). In other embodiments, the therapeutically effective amount of CB1 antagonist is lower than the minimum dose of the CB1 antagonist proven effective in a United States clinical trial of the CB1 antagonist, wherein the trial is conducted without coadministration of a second anti-obesity agent, such as a MCHR antagonist (e.g., the therapeutically effective amount is less than 95%, less than 90%, less than 75% or less than 50% of the minimum dose proven effective in such a clinical trial). In still further such embodiments, both the MCHR antagonist and the CB1 antagonist are employed at doses that are lower than the minimum dose proven effective in such clinical trials. The phrase “clinical trial,” as used herein, refers to an experimental study in human subjects performed for purposes related to the development and submission of information under a federal law which regulates the manufacture, use or sale of drugs.

In other embodiments, the therapeutically effective amount of MCHR antagonist is lower than the minimum marketed dose (for the patient's size) for use without coadministration of a second anti-obesity agent and/or the therapeutically effective amount of CB1 antagonist is lower than the minimum marketed dose (for the patient's size) for use without coadministration of a second anti-obesity agent. For example, the therapeutically effective amount of one or both of MCHR antagonist and CB1 antagonist may be less than 95%, less than 90%, less than 75% or less than 50% of the minimum marketed dose. In certain such embodiments, the patient is a non-human animal, such as a companion animal (e.g., a dog or cat).

Packaged pharmaceutical preparations provided herein generally include a container holding a composition comprising at least one MCHR antagonist and instructions (e.g., labeling) indicating that the contained composition is to be used in a therapeutically effective amount for reducing appetite or treating obesity in a patient. In certain embodiments, the instructions further specify that the contained composition is to be used in combination with a therapeutically effective amount of CB1 antagonist. In other embodiments, the packaged pharmaceutical preparation further comprises one or more CB1 antagonists in the same container or in a separate container within the package. Preferred mixtures are formulated for oral administration (e.g., as pills, capsules, tablets or the like). Therapeutically effective amounts for use in packaged pharmaceutical preparations are generally as discussed above.

Therapeutic Methods

Within certain aspects, the present invention provides methods that are useful for weight management. Within such methods, a composition as provided herein is administered to a patient in order to reduce appetite and/or food intake or to prevent or treat obesity (e.g., to promote weight loss). Patients may include humans, domesticated companion animals (pets, such as dogs) and livestock animals, with dosages and treatment regimes as described above.

The present invention provides methods for using one or more MCHR antagonists in combination with one or more CB1 antagonists for weight management. The MCHR antagonist(s) may be administered to the patient at the same time as the CB 1 antagonist(s) (e.g., as a single dosage unit), or may be administered separately (before or after CB1 antagonist). Within preferred embodiments, the MCHR antagonist(s) and CB1 antagonist(s) are ultimately simultaneously present at effective concentrations in a body fluid (e.g., blood) of the patient. An effective concentration of MCHR antagonist or CB1 antagonist is a concentration that is sufficient to reduce one or more of food consumption, appetite and/or body mass index in the patient when repeatedly coadministered as described herein. As noted above, an effective concentration of MCHR antagonist may be achieved by administration of a therapeutically effective amount of MCHR antagonist that is lower than the amount that would need to be administered to effect a comparable patient benefit in the absence of CB1 antagonist and/or an effective concentration of CB1 antagonist may be achieved by administration of a therapeutically effective amount of CB1 antagonist that is lower than the amount that would need to be administered to effect a comparable patient benefit in the absence of MCHR antagonist.

Therapeutically effective amounts for use in such methods are generally as described above. In certain embodiments, the therapeutically effective amount of one or both of MCHR antagonist and CB1 antagonist is less than ¾, ½, ¼ or 10% of the maximum recommended dose as described above. In other embodiments, the therapeutically effective amount of one or both of MCHR antagonist and CB1 antagonist is less than the minimum dose proven effective in a United States clinical trial. In further embodiments, the therapeutically effective amount of one or both of MCHR antagonist and CB1 antagonist is lower than the minimum marketed dose for the size of the patient treated.

Administration of the MCHR antagonist and CB1 antagonist to the patient can be by way of any means discussed above, including oral, topical, nasal or transdermal administration, or intravenous, intramuscular, subcutaneous, intrathecal, epidural, intracerebroventricular or like injection. In certain embodiments, a mixture of one or more MCHR antagonists and one or more CB1 antagonists, as described above, is administered. Preferred mixtures for use within such methods are formulated for oral administration (e.g., as pills, capsules, tablets or the like) or intravenous administration.

Frequency of dosage may vary depending on the compound used and the level of obesity in the patient. In general, a dosage regimen of 4 times daily or less is preferred, with I or 2 times daily particularly preferred. The specific dose for any particular patient will depend upon a variety of factors, as discussed above. Patients may generally be monitored for therapeutic effectiveness using assays suitable for the condition being treated or prevented, which will be familiar to those of ordinary skill in the art. For example, treatment is considered to be effective if it results in a statistically significant decrease in weight, BMI or food intake.

Methods for Identifying MCHR Antagonist/CB1 Antagonist Combinations

The present invention further provides methods for selecting MCHR antagonist/CB1 antagonist combinations for use in the pharmaceutical compositions, packaged pharmaceutical preparations and methods described above. Such methods are generally based on the selection of combinations in which the MCHR antagonist and CB1 antagonist display at least an additive effect on food intake in a mammal. For example, the present invention provides methods for identifying a MCHR antagonist / CB1 antagonist combination for use in the regulation of body weight, the method comprising identifying a MCHR antagonist and a CB1 antagonist that exhibit at least an additive effect on food intake in a mammal when administered contemporaneously to the mammal in therapeutically effective amounts, and therefrom identifying a MCHR antagonist/CB1 antagonist combination for use in the regulation of body weight Methods are further provided for selecting active ingredients for a pharmaceutical composition or for selecting therapeutic agents for coadministration to a patient generally comprise selecting a MCHR antagonist and a CB1 antagonist that exhibit at least an additive effect on food intake in a mammal when administered contemporaneously to the mammal in therapeutically effective amounts. Within certain embodiments, methods for selecting active ingredients for a pharmaceutical composition comprise the steps of: (i) determining an effect of a MCHR antagonist on food intake in a mammal (e.g., a non-human mammal) treated with a first therapeutically effective amount of the MCHR antagonist without coadministration of CB1 antagonist; (ii) determining an effect of a CB1 antagonist on food intake in a mammal treated with a second therapeutically effective amount of the CB1 antagonist without coadministration of MCHR antagonist; (iii) determining an effect of the MCHR antagonist and the CB1 antagonist on food intake in a mammal treated contemporaneously with the first therapeutically effective amount of the MCHR antagonist and the second therapeutically effective amount of the CB1 antagonist; (iv) optionally repeating steps (i)-(iii) with a different MCHR antagonist or a different CB1 antagonist; and (v) selecting a MCHR antagonist and a CB1 antagonist for which the effect on food intake determined in step (iii) is equal to or greater than the sum of the effects on food intake determined in steps (i) and (ii).

Methods for preparing a pharmaceutical composition comprising such active ingredients generally comprises the steps of: (i) identifying a MCHR antagonist and a CB1 antagonist that have at least an additive effect on food intake in a mammal when administered contemporaneously to the mammal in therapeutically effective amounts; and (ii) combining a therapeutically effective amount of the MCHR antagonist and a therapeutically effective amount of the CB1 antagonist with a physiologically acceptable carrier or excipient. Methods for preparing a packaged pharmaceutical preparation comprising such active ingredients generally comprise the steps of: (i) identifying a MCHR antagonist and a CB1 antagonist that have at least an additive effect on food intake in a mammal when administered contemporaneously to the mammal in therapeutically effective amounts; and (ii) packaging the MCHR antagonist and the CB1 antagonist with instructions indicating that a first therapeutically effective amount of MCHR antagonist and a second therapeutically effective amount of CB1 antagonist are to be administered contemporaneously to a patient for reducing food intake or appetite or for treating obesity.

The following Examples are offered by way of illustration and not by way of limitation. Unless otherwise specified all reagents and solvents are of standard commercial grade and are used without further purification.

### EXAMPLES

### Example 1

### Effect of MCHR Antagonist and CB1 Antagonist on Food Consumption

This Example illustrates an in vivo assay that measures food intake and body weight change over a 12 hour period in satiated rats. This assay is used for use to confirm the ability of coadministration of a MCHR antagonist and CB1 antagonist to inhibit excess food consumption.

Rats (3 months old, 250-300 g) are weighed. Prior to the assay, the rats are provided with ad libitum access to normal chow and water. Control and test compound solutions are then administered orally, and after 30 minutes the rats are placed in metabolic feeding cages (Nalgene metabolic cages, VWR Scientific) for a 12 hour feeding session, with lights out, during which time ad libitum access to food and water is provided. The food provided is a palatable chow diet essentially as described by Tritos et al., Diabetes 47:1687 (1998). After 12 hours, food intake, water intake, urine and feces output and body weight change are measured.

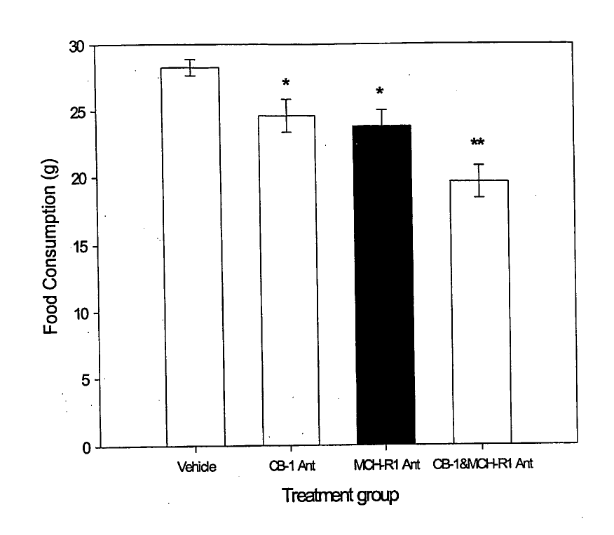

FIG. 1 shows the results of one such assay, in which animals are divided into 4 treatment groups, with 8-10 animals per group. In the first group, the rats are treated with vehicle alone (5% methylcellulose with 0.1% triacetin, a wetting agent). These vehicle-treated animals eat an average of 28 g of food during the 12 hour feeding session. In the second group, animals are treated with one dose of 5 mg/kg CB1 antagonist. These animals show an average reduction in food intake of 13%. The third group of animals is treated with one dose of 10 mg/kg MCHR1 antagonist. These animals show an average reduction in food intake of 16%. In the fourth group, animals are treated with both the CB1 antagonist and the MCHR1 antagonist, as described above, and display a 31% reduction in food intake. This result demonstrates that coadministration of these two agents provides at least an additive reduction in food intake.

### Example 2

### Melanin Concentrating Hormone Receptor Binding Assay

This Example illustrates a standard assay of MCH receptor binding that may be used to determine the binding affinity of compounds for the MCH receptor.

Cynomolgus macaque hypothalamus MCHR1 cDNA is prepared and cloned into PCDNA3.1 (INVITROGEN Corp., Carlsbad, Calif.), and HEK293 cells (American Type Culture Collection, Manassas, Va.) are stably transfected with the MCHR1 expression vector as described in PCT International Application publication number WO 03/059289, which published on Jul. 24, 2003. The disclosure of WO 03/059289 at page 52 directed to the preparation and storage of the transfected HEK293 cells is hereby incorporated by reference.

At the time of assay, HEK293 cell membrane pellets are thawed by addition of wash buffer (25 mM HEPES with 1.0 nM CaCl₂, 5.0 mM MgCl₂, 120 mM NaCl, pH 7.4) and homogenized for 30 seconds using a BRINKMAN POLYTRON, setting 5. Cells are centrifuged for 10 minutes at 48,000×g. The supernatant is discarded and the pellet is resuspended in fresh wash buffer, and homogenized again. An aliquot of this membrane homogenate is used to determine protein concentration via the Bradford method (BIO-RAD Protein Assay Kit, #500-0001, BIO-RAD, Hercules, Calif.). By this measure, a 1-liter culture of cells typically yields 50-75 mg of total membrane protein. The homogenate is centrifuged as before and resuspended to a protein concentration of 333 μg/mL in binding buffer (Wash buffer +0.1% BSA and 1.0 μM final phosphoramidon) for an assay volume of 50 μg membrane protein/150 μL binding buffer. Phosphoramidon was from SIGMA BIOCHEMICALS, St. Louis, Mo. (cat# R-7385).

Competition binding assays are performed at room temperature in Falcon 96 well round bottom polypropylene plates. Each assay well contains 150 μL of MCH receptor-containing membranes prepared as described above, 50 μL ¹²⁵I-Tyr MCH, 50 μL binding buffer, and 2 μL test compound in DMSO. ¹²⁵I-Tyr MCH (specific activity=2200 Ci/mmol) is purchased from NEN, Boston, Mass. (Cat # NEX 373) and is diluted in binding buffer to provide a final assay concentration of 30 pM.

Non-specific binding is defined as the binding measured in the presence of 1 μM unlabeled MCH. MCH is purchased from BACHEM U.S.A., King of Prussia, Pa. (cat # H-1482). Assay wells used to determine MCH binding contain 150 μL of MCH receptor containing membranes, 50 μL ¹²⁵I-Tyr MCH, 25 μL binding buffer and 25 μL binding buffer.

Assay plates are incubated for 1 hour at room temperature. Membranes are harvested onto WALLAC™ glass fiber filters (PERKIN-ELMER, Gaithersburg, Md.) which were pre-soaked with 1.0% PEI (polyethyleneimine) for 2 hours prior to use. Filters are allowed to dry overnight, and then counted in a WALLAC 1205 BETA PLATE counter after addition of WALLAC BETA SCIN™ scintillation fluid.

For saturation binding, the concentration of ¹²⁵I-Tyr MCH is varied from 7 to 1,000 pM. Typically, 11 concentration points are collected per saturation binding curve. Equilibrium binding parameters are determined by fitting the Hill equation to the measured values with the aid of the computer program Fit™ (BIOSOFT, Ferguson, Mo.). For preferred MCHR antagonists, Kᵢ values are below 1 micromolar, preferably below 500 nanomolar, more preferably below 100 nanomolar.

### Example 3

### MCH Receptor Calcium Mobilization Assay

This Example illustrates a representative functional assay for monitoring the response of cells expressing melanin concentrating hormone receptors to melanin concentrating hormone. This assay can also be used to determine if test compounds act as agonists or antagonists of melanin concentrating hormone receptors.

Chinese Hamster Ovary (CHO) cells (American Type Culture Collection; Manassas, Va.) are stably transfected with the MCH receptor expression vector via calcium phosphate precipitation. The best expressing transfectant is chosen, and this transfectant (i.e., clone) is amplified and grown to a density of 15,000 cells/well in FALCON™ black-walled, clear-bottomed 96-well plates (#3904, BECTON-DICKINSON, Franklin Lakes, N.J.) in Ham's F12 culture medium (MEDIATECH, Hemdon, Va.) supplemented with 10% fetal bovine serum, 25 mM HEPES and 500 μg/mL (active) G418. Prior to running the assay, the culture medium is emptied from the 96 well plates. Fluo-3 calcium sensitive dye (Molecular Probes, Eugene, OR) is added to each well (dye solution: 1 mg FLUO-3 AM, 440 μL DMSO and 440 μL 20% pluronic acid in DMSO, diluted 1:4, 50 μL diluted solution per well). Plates are covered with aluminum foil and incubated at 37° C. for 1-2 hours. After the incubation, the dye is emptied from the plates, cells are washed once in 100 μL KRH buffer (0.05 nM KCl, 0.115 M NaCl, 9.6 mM NaH₂PO₄, 0.01 nM MgSO₄, 25 mM HEPES, pH 7.4) to remove excess dye; after washing, 80 μL KRH buffer is added to each well.

Fluorescence response is monitored upon the addition of either human MCH receptor or test compound by a FLIPR™ plate reader (Molecular Devices, Sunnyvale, Calif.) by excitation at 480 nm and emission at 530 mn.

In order to measure the ability of a test compound to antagonize the response of cells expressing MCH receptors to MCH, the EC₅₀ of MCH is first determined. An additional 20 μL of KRH buffer and 1 μL DMSO is added to each well of cells, prepared as described above. 100 μL human MCH in KRH buffer is automatically transferred by the FLIPR instrument to each well. An 8-point concentration response curve, with final MCH concentrations of 1 nM to 3 μM, is used to determine MCH EC₅₀.

Test compounds are dissolved in DMSO, diluted in 20 μL KRH buffer, and added to cells prepared as described above. The 96 well plates containing prepared cells and test compounds are incubated in the dark, at room temperature for 0.5-6 hours. It is important that the incubation not continue beyond 6 hours. Just prior to determining the fluorescence response, 100 μL human MCH diluted in KRH buffer to 2×EC₅₀ is automatically added by the FLIPR instrument to each well of the 96 well plate for a final sample volume of 200 μL and a final MCH concentration of EC₅₀. The final concentration of test compounds in the assay wells is between 1 nM and 5 nM. Typically, cells exposed to one EC₅₀ of MCH exhibit a fluorescence response of about 10,000 Relative Fluorescence Units. Cells incubated with antagonists of the MCH receptor exhibit a response that is significantly less than that of the control cells to the p≦0.05 level, as measured using a parametric test of statistical significance. Typically, antagonists of the MCH receptor decrease the fluorescence response by about 20%, preferably by about 50%, and most preferably by at least 80% as compared to matched controls. IC₅₀ values for MCHR antagonists are determined using SIGMAPLOT software (SPSS Inc., Chicago, Ill.) and standard techniques. The IC₅₀ is then used to generate Kᵢ as described by Cheng and Prusoff (1973) Biochem Pharmacol. 22(23):3099-108.

The ability of a compound to act as an agonist of the MCH receptor is determined by measuring the fluorescence response of cells expressing MCH receptors, using the methods described above, in the absence of MCH. Compounds that cause cells to exhibit fluorescence above background are MCH receptor agonists (background autofluorescence of the test compound may be assessed using standard methods). MCHR antagonists that induce no detectable increase in the basal activity of the MCH receptor have no detectable agonist activity and are preferred.

### Example 4

### Baculoviral Preparations for CB1 Expression

This Example illustrates the preparation of recombinant baculovirus for use in generating CB1-expressing insect cells.

The human CB1 sequence has GenBank Accession Number HSU73304, and was reported by Hoehe et al. (1991) New Biol. 3(9):880-85. Human CB1 (hCB1) cDNA is amplified from a human brain cDNA library (Gibco BRL, Gaithersburg, Md.) using PCR, in which the 5′ primer includes the optimal Kozak sequence CCACC. The resulting PCR product is cloned into pcDNA3.1/V5-His-TOPO (Invitrogen Corp, Carlsbad, Calif.) using the multiple cloning site, and then subcloned into pBACPAK₈ (BD Biosciences, Palo Alto, Calif.) at the Barn/Xho site to yield a hCB1 baculoviral expression vector.

The hCB1 baculoviral expression vector is co-transfected along with BACULOGOLD DNA (BD PharMingen, San Diego, Calif.) into Sf9 cells. The Sf9 cell culture supernatant is harvested three days post-transfection. The recombinant virus-containing supernatant is serially diluted in Hink's TNM-FH insect medium (JRH Biosciences, Kansas City, Mo.) supplemented with Grace's salts and with 4.1 mM L-Gln, 3.3 g/L LAH, 3.3 g/L ultrafiltered yeastolate and 10% heat-inactivated fetal bovine serum (hereinafter “insect medium”) and plaque assayed for recombinant plaques. After four days, recombinant plaques are selected and harvested into 1 ml of insect medium for amplification. Each 1 ml volume of recombinant baculovirus (at passage 0) is used to infect a separate T25 flask containing 2×10⁶ Sf9 cells in 5 ml of insect medium. After five days of incubation at 27° C., supernatant medium is harvested from each of the T25 infections for use as passage 1 inoculum.

Two of seven recombinant baculoviral clones are then chosen for a second round of amplification, using 1 ml of passage 1 stock to infect 1×10⁸ cells in 100 ml of insect medium divided into 2 T175 flasks. Forty-eight hours post infection, passage 2 medium from each 100 ml prep is harvested and plaque assayed for titer. The cell pellets from the second round of amplification are assayed by affinity binding as described below to verify recombinant receptor expression. A third round of amplification is then initiated using a multiplicity of infection of 0.1 to infect a liter of Sf9 cells. Seventy-two hours post-infection the supernatant medium is harvested to yield passage 3 baculoviral stock.

The remaining cell pellet is assayed for affinity binding. Radioligand is 25 pM-5.0 nM [³H]CP55,940 for saturation binding and 0.5 nM for competition binding (New England Nuclear Corp., Boston, Mass.); the hCB1-expressing baculoviral cells are used; the assay buffer contains 50 mM Tris pH 7.4, 120 mM NaCl, 5 mM MgCl₂, 0.5% BSA and 0.2 mg/ml bacitracin; filtration is carried out using GF/C WHATMAN filters (presoaked in 0.3% non-fat dry milk (H₂O) for 2 hours prior to use); and the filters are washed twice with 5 mL cold 50 mM Tris pH. 7.4.

Titer of the passage 3 baculoviral stock is determined by plaque assay and a multiplicity of infection, incubation time course, binding assay experiment is carried out to determine conditions for optimal receptor expression.

### Example 5

### Baculoviral Infections

Log-phase Sf9 cells (INVITROGEN Corp., Carlsbad Calif.), are infected with one or more stocks of recombinant baculovirus followed by culturing in insect medium at 27″C. Infections are carried out either only with virus directing the expression of hCB1 or with this virus in combination with three G-protein subunit-expression virus stocks: 1) rat Gα₂ G-protein-encoding virus stock (BIOSIGNAL #V5J008), 2) bovine β1 G-protein-encoding virus stock (BIOSIGNAL #V5H012), and 3) human γ2 G-protein-encoding virus stock (BIOSIGNAL #V6B003), all of which are obtained from BIOSIGNAL Inc., Montreal, Canada.

Typical hCB1 infections are conducted using Sf9 cells that are cultured in insect medium supplemented with 10% heat-inactivated fetal bovine serum (FBS) as discussed above. Higher receptor and G-protein (Gα, Gβ, Gγ) expression can be obtained if the Sf9 cells are cultured in insect medium with 5% FBS and 5% Gibco serum-free medium (INVITROGEN Corp., Carlsbad Calif.). Maximal CB1 receptor expression and functional activity is achieved if the Sf9 cells are cultured in insect medium without FBS and with 10% Gibco serum-free medium. The infections are carried out at a multiplicity of infection of 0.1:1.0:0.5:0.5. At 72 hours post-infection, a sample of cell suspension is analyzed for viability by trypan blue dye exclusion, and the remaining Sf9 cells are harvested via centrifugation (3000 rpm/10 minutes/4° C.).

### Example 6

### Purified Recombinant Insect Cell Membranes

Sf9 cell pellets are resuspended in homogenization buffer (10 mM HEPES, 250 mM sucrose, 0.5 μg/ml leupeptin, 2 μg/ml Aprotinin, 200 μM PMSF, and 2.5 mM EDTA, pH 7.4) and homogenized using a POLYTRON homogenizer (setting 5 for 30 seconds). The homogenate is centrifuged (536×g/10 minutes/4° C.) to pellet the nuclei. The supernatant containing isolated membranes is decanted to a clean centrifuge tube, centrifuged (48,000×g/30 minutes, 4° C.) and the resulting pellet resuspended in 30 ml homogenization buffer. This centrifugation and resuspension step is repeated twice. The final pellet is resuspended in ice cold Dulbecco's PBS containing 5 mM EDTA and stored in frozen aliquots at −80° C. until needed. The protein concentration of the resulting membrane preparation (hereinafter “P2 membranes”) is measured using a Bradford protein assay (Bio-Rad Laboratories, Hercules, Calif.). By this measure, a 1-liter culture of cells typically yields 100-150 mg of total membrane protein.

### Example 7

### Radioligand Binding Assays

P2 membranes are resuspended by Dounce homogenization (tight pestle) in binding buffer (50 mM Tris pH. 7.4, 120nM NaCl, 5 mM MgCl₂, 0.5% BSA and 0.2 mg/ml bacitracin).

For saturation binding analysis, membranes (10 μg) are added to polypropylene tubes containing 25 pM-0.5 nM [³H]CP55,940 (New England Nuclear Corp., Boston, Mass.). Nonspecific binding is determined in the presence of 10 μM CP55,940 (Tocris Cookson Inc., Ellisville, Mo.) and accounted for less than 10% of total binding. For evaluation of guanine nucleotide effects on receptor affinity, GTPγS is added to duplicate tubes at the final concentration of 50 μM.

For competition analysis, membranes (10 μg) are added to polypropylene tubes containing 0.5 nM [³H]CP55,940. Non-radiolabeled displacers are added to separate assays at concentrations ranging from 10.10 M to 10′5 M to yield a final volume of 0.250 mL. Nonspecific binding is determined in the presence of 10AM CP55,940 and accounted for less than 10% of total binding. Following a 1-hour incubation at room temperature, the reaction is terminated by rapid vacuum filtration. Samples are filtered over presoaked (0.3% non-fat dry milk for 2 hours prior to use) GF/C WHATMAN filters and rinsed 2 times with 5 mL cold 50 nm Tris pH 7.4. Remaining bound radioactivity is quantified by gamma counting. Kᵢ and Hill coefficient (“nH”) are determined by fitting the Hill equation to the measured values with the aid of SIGMAPLOT software.

### Example 8

### Agonist-Induced GTP Binding

This Example illustrates the use of agonist-stimulated GTP-gamma³⁵S binding (“GTP binding”) activity to identify CB1 agonists and antagonists, and to differentiate neutral antagonists from those that possess inverse agonist activity. This assay can also be used to detect partial agonism mediated by antagonist compounds. A compound being analyzed in this assay is referred to herein as a “test compound.” Agonist-stimulated GTP binding activity is measured as follows: Four independent baculoviral stocks (one directing the expression of hCB1 and three directing the expression of each of the three subunits of a heterotrimeric G-protein) are used to infect a culture of Sf9 cells as described in Example 5.

Agonist-stimulated GTP binding on purified membranes (prepared as described in Example 6) is initially assessed using the CB1 agonist CP55,940 to ascertain that the receptor/G-protein-alpha-beta-gamma combination(s) yield a functional response as measured by GTP binding.

P2 membranes are resuspended by Dounce homogenization (tight pestle) in GTF binding assay buffer (50 mM Tris pH 7.4, 120 mM NaCl, 5 mM MgCl₂, 2 mM EGTA, 0.1% BSA, 0.1 mM bacitracin, 100 KIU/mL aprotinin, 5 μM GDP) and added to reaction tubes at a concentration of 10 μg protein/reaction tube. After adding increasing doses of the agonist CP55,940 at concentrations ranging from 10⁻¹² M to 10⁻⁶ M, reactions are initiated by the addition of 100 pM GTP-gamma³⁵S. In competition experiments, non-radiolabeled test compounds are added to separate assays at concentrations ranging from 10⁻¹⁰ M to 10⁻⁵ M along with 1 nM CP55,940 to yield a final volume of 0.25 mL.

Following a 60-minute incubation at room temperature, the reactions are terminated by vacuum filtration over GF/C filters (pre-soaked in wash buffer, 0.1% BSA) followed by washing with ice-cold wash buffer (50 mM Tris pH 7.0, 120 mM NaCl). The amount of receptor-bound (and thereby membrane-bound) GTP-gamma³⁵S is determined by measuring the bound radioactivity, preferably by liquid scintillation spectrometry of the washed filters. Non-specific binding is determined using 10 mM GTP-gamma³⁵S and typically represents less than 5 percent of total binding. Data is expressed as percent above basal (baseline). The results of these GTP binding experiments are analyzed using SIGMAPLOT software and IC₅₀ determined. The IC₅₀ is then used to generate Kᵢ as described by Cheng and Prusoff (1973) Biochem Pharmacol. 22(23):3099-108.

Neutral antagonists are those test compounds that reduce the CP55,940-stimulated GTP binding activity towards, but not below, baseline (the level of GTP bound by membranes in this assay in the absence of added CP55,940 or other agonist and in the further absence of any test compound).

In contrast, in the absence of added CP55,940, CB1 inverse agonists reduce the GTP binding activity of the receptor-containing membranes below baseline. If a test compound that displays antagonist activity does not reduce the GTP binding activity below baseline in the absence of the CB1 agonist, it is characterized as a neutral antagonist.

An antagonist test compound that elevates GTP binding activity above baseline in the absence of added CP55,940 in this GTP binding assay is characterized as having partial agonist activity. Preferred CB1 antagonists do not elevate GTP binding activity under such conditions more than 10%, more preferably less than 5% and most preferably less than 2% of the maximal response elicited by the agonist, CP55,940.

### Example 9

### MDCK Cytotoxicity Assay

This Example illustrates the evaluation of compound toxicity using a Madin Darby canine kidney (MDCK) cell cytotoxicity assay.

1 μL of test compound is added to each well of a clear bottom 96-well plate (PACKARD, Meriden, Conn.) to give final concentration of compound in the assay of 10 μM, 100 μM or 200 μM. Solvent without test compound is added to control wells.

MDCK cells, ATCC no. CCL-34 (American Type Culture Collection, Manassas, Va.), are maintained in sterile conditions following the instructions in the ATCC production information sheet. Confluent MDCK cells are trypsinized, harvested, and diluted to a concentration of 0.1×10⁶ cells/mL with warm (37° C.) medium (VITACELL Minimum Essential Medium Eagle, ATCC catalog # 30-2003). 100 μL of diluted cells is added to each well, except for five standard curve control wells that contain 100 μL of warm medium without cells. The plate is then incubated at 37° C. under 95% O₂, 5% CO₂ for 2 hours with constant shaking. After incubation, 50 μL of mammalian cell lysis solution (from the PACKARD (Meriden, CT) ATP-LITE-M Luminescent ATP detection kit) is added per well, the wells are covered with PACKARD TOPSEAL stickers, and plates are shaken at approximately. 700 rpm on a suitable shaker for 2 minutes.

Compounds causing toxicity will decrease ATP production, relative to untreated cells. The ATP-LITE-M Luminescent ATP detection kit is generally used according to the manufacturer's instructions to measure ATP production in treated and untreated MDCK cells. PACKARD ATP LITE-M reagents are allowed to equilibrate to room temperature. Once equilibrated, the lyophilized substrate solution is reconstituted in 5.5 mL of substrate buffer solution (from kit). Lyophilized ATP standard solution is reconstituted in deionized water to give a 10 nM stock. For the five control wells, 10 μL of serially diluted PACKARD standard is added to each of the standard curve control wells to yield a final concentration in each subsequent well of 200 nM, 100 nM, 50 nM, 25 nM, and 12.5 nM. PACKARD substrate solution (50 μL) is added to all wells, which are then covered, and the plates are shaken at approximately 700 rpm on a suitable shaker for 2 minutes. A white PACKARD sticker is attached to the bottom of each plate and samples are dark adapted by wrapping plates in foil and placing in the dark for 10 minutes. Luminescence is then measured at 22° C. using a luminescence counter (e.g., PACKARD TOPCOUNT Microplate Scintillation and Luminescence Counter or TECAN SPECTRAFLUOR PLUS), and ATP levels calculated from the standard curve. ATP levels in cells treated with test compound(s) are compared to the levels determined for untreated cells. Cells treated with 10 μM of a preferred test compound exhibit ATP levels that are at least 80%, preferably at least 90%, of the untreated cells. When a 100 μM concentration of the test compound is used, cells treated with preferred test compounds exhibit ATP levels that are at least 50%, preferably at least 80%, of the ATP levels detected in untreated cells.

1. A pharmaceutical composition comprising a first therapeutically effective amount of a nontoxic MCHR antagonist and a second therapeutically effective amount of a nontoxic CB1 antagonist, together with a physiologically acceptable carrier or excipient, wherein the first therapeutically effective amount is less than ½ the maximum recommended dose for the MCHR antagonist.

2. The composition of claim 1, wherein first therapeutically effective amount is less than ¼ the maximum recommended dose for the MCHR antagonist.

3. The composition of claim 2, wherein the first therapeutically effective amount is less than 10% of the maximum recommended dose for the MCHR antagonist.

4. The composition of claim 1, wherein the second therapeutically effective amount is less than ½ the maximum recommended dose for the CB1 antagonist.

5. A pharmaceutical composition comprising a first therapeutically effective amount of a nontoxic MCHR antagonist and a second therapeutically effective amount of a nontoxic CB1 antagonist, together with a physiologically acceptable carrier or excipient, wherein the second therapeutically effective amount is less than ½ the maximum recommended dose for the CB1 antagonist.

6. The composition of claim 5, wherein the second therapeutically effective amount is less than ¼ the maximum recommended dose for the CB1 antagonist.

7. The composition of claim 6, wherein the second therapeutically effective amount is less than 10% of the maximum recommended dose for the CB1 antagonist.

8. The composition of claim 1, wherein the MCHR antagonist has no detectable MCH receptor agonist activity.

9. The composition of claim 1, wherein the MCHR antagonist is a MCHR1 antagonist.

10. The composition of claim 1 in sustained release dosage form.

11. The composition of claim 1 formulated for oral administration.

12-33. (canceled)

34. A method for reducing appetite or food intake in a patient, comprising contemporaneously administering to a patient:
(i) a first therapeutically effective amount of a nontoxic MCHR antagonist; and
(ii) a second therapeutically effective amount of a nontoxic CB1 antagonist;
wherein the first therapeutically effective amount is less than ½ the maximum recommended dose for the MCHR antagonist;
and thereby reducing appetite or food intake in the patient.

35-36. (canceled)

37. The method of claim 34, wherein the second therapeutically effective amount is less than ½ the maximum recommended dose for the CB1 antagonist.

38. A method for reducing appetite or food intake in a patient, comprising contemporaneously administering to a patient:
(i) a first therapeutically effective amount of a nontoxic MCHR antagonist; and
(ii) a second therapeutically effective amount of a nontoxic CB1 antagonist;
wherein the second therapeutically effective amount is less than ½ the maximum recommended dose for the CB1 antagonist;
and thereby reducing appetite or food intake in the patient.

39. (canceled)

40. The method of claim 38, wherein the second therapeutically effective amount is less than 10% of the maximum recommended dose for the CB1 antagonist.

41-104. (canceled)

105. A method for identifying therapeutic agents for coadministration to a patient, the method comprising selecting a MCHR antagonist and a CB1 antagonist that exhibit at least an additive effect on food intake in a mammal when administered contemporaneously to the mammal in therapeutically effective amounts, and therefrom identifying therapeutic agents for coadministration to the patient.

106-107. (canceled)

108. A method for determining CB1 antagonist activity of a test compound, comprising:
(a) contacting a first cell membrane preparation comprising CB1 with:
(i) labeled GTP;
(ii) a CB1 agonist; and
(iii) a test compound; to yield a test membrane preparation;
(b) contacting a second cell membrane preparation comprising CB1 with:
(i) labeled GTP; and
(ii) a CB1 agonist; to yield a control membrane preparation;
wherein steps (a) and (b) are performed simultaneously or in either order and under conditions suitable for GTP binding to the CB1;
(c) detecting, simultaneously or in either order:
(i) a test signal that represents an amount of bound, labeled GTP in the test membrane preparation; and
(ii) a control signal that represents an amount of bound, labeled GTP in the control membrane preparation; and
(d) comparing the test signal with the control signal;
and therefrom determining CB1 antagonist activity of the test compound.

109-110. (canceled)

111. The composition of claim 5, wherein the MCHR antagonist has no detectable MCH receptor agonist activity.

112. The composition of claim 5, wherein the MCHR antagonist is a MCHR1 antagonist.

113. The composition of claim 5, in sustained release dosage form.

114. The composition of claim 5, formulated for oral administration.
